SEQUENCE LISTING (4 sequences)

SEQ ID NO: 1 - protein, 12 aa, Artificial
  Synthetic peptide
EEAEEAEEAAPF

SEQ ID NO: 2 - protein, 9 aa, Artificial
  synthetic peptide
CAGSGSGPR

SEQ ID NO: 3 - DNA, 56 bp, Artificial
  Synthetic oligonucleotide
tttttgaattctaatacgactcagaatgagtctgggcctctttttaagaacttttt

SEQ ID NO: 4 - protein, 6 aa, Artificial
  Synthetic peptide
GAAAPF

(19) United States
(12) Patent Application Publication
(10) Pub. No.: US 2007/0111222 A1
(43) Pub. Date: May 17, 2007

(54) Nucleic acid aptamer-based compositions and methods

(75) Inventors: Lawrence Chasin, Leonia, NJ (US); Ponisseril Somasundaran, Nyack, NY (US); Collin Nuckolls, New York, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/438,758

(22) Filed: May 22, 2006

Related U.S. Application Data
(63) Continuation-in-part of application No. PCT/US04/39329, filed on Nov. 19, 2004.
(60) Provisional application No. 60/524,740, filed on Nov. 21, 2003.

(51) Int. Cl. A61K 48/00; A61K 38/28; C12Q 1/68; C12M 3/00

(52) U.S. Cl. 435/6; 435/287.2; 514/3; 514/44; 977/924

(57) ABSTRACT

The present invention relates to compositions that can detect the presence of specific entities or substances in an environment, and provide an amplified response to the detection as manifested by release of enzymes, reporter signals or drugs. The detection and response is based on nucleic acid functionalities, such as aptamer regions that are designed to specifically bind almost any entity or ligand, and enzymatic regions that can cleave nucleic acids at specific sequences. The response can be amplified on a first order through creating an allosteric relationship between the different nucleic acid functionalities present on the same nucleic acid molecule and on a second order through the release of active cargo molecules capable of generating molecules detectable by their color, fluorescence, luminescence, or ability to modulate an electric signal.

This application is a continuation-in-part of PCT/US04/39329, which was filed on Nov. 19, 2004 and claims priority to U.S. Ser. No. 60/524,740, which was filed on Nov. 21, 2003, both of which are hereby incorporated by reference in their entireties.

The invention disclosed herein was made with U.S. Government support from the National Science Foundation (NSF SGER CTS-03-4694). Accordingly, the U.S. Government has certain rights in this invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

Single stranded and double stranded nucleic acids can adopt complex three-dimensional conformations that exhibit specific binding abilities and even enzymatic activities. While proteins can also exhibit these characteristics, the ability of nucleic acids to be chemically synthesized inexpensively and enzymatically amplified makes them proficient for sensing and responding to molecular elements.

In particular, nucleic acids can be synthesized to have aptamer domains which allow the specific detection and response to molecular ligands. Aptamers are oligonucleotides that bind to a particular ligand with great affinity and selectivity. The ligands can range from metal ions to small organic molecules, to proteins, to supramolecular entities, to viruses and to bacteria. Further, it has been reported that the binding ability of an aptamer domain could be modulated by a second aptamer domain residing on the same oligonucleotide. When an aptamer domain binds a ligand it can effect a conformational change in the whole molecule that either activates or inactivates the second aptamer.

This allosteric relationship can also be observed between an aptamer domain and other nucleic acid functional domains. For example, upon binding of a ligand to an aptamer domain, the binding can cause a conformational change in the nucleic acid such that the catalytic activity of a ribozyme or a DNAzyme functional domain present on the same nucleic acid becomes activated. Nucleic acid functional domains, such as aptamers, ribozymes, and DNAzymes can be specifically synthesized to recognize specific ligands (for aptamers) or to cleave at specific sequences (for ribozymes and DNAzymes) through selection and amplification. Allosteric control between different nucleic acid domains can be selected for in much the same way as simple ligand binding, since the individual aptamer domains can act as semi-autonomous modules. However, these reports do not solve the problem of providing an amplified response to low levels of ligand, where a response is often too weak for practical applications due to a linear relationship between the ligand levels and the response. Further, these reports do not mention the use of nucleic acid functionalities to provide a controlled drug delivery method.

### SUMMARY OF THE INVENTION

The present invention relates to compositions that can detect the presence of specific entities or substances in an environment. The compositions then provide an amplified response to the detection of the substance by release of a specified molecule. The present compositions are therefore used in methods for detecting environmental entities, such as bioterrorism agents, or for detecting physiological agents as the basis of a controlled drug delivery.

The compositions of the present invention are designed in consideration of several nucleic acid functionalities. For example, the present invention is useful to detect entities in any environment (in vivo, in vitro, natural or man-made) through the use of nucleic acids containing aptamer regions, where the aptamer regions are designed to specifically bind almost any entity or ligand, including single molecules, small-molecules, proteins, supramolecular entities, and microorganisms such as viruses and bacteria.

The nucleic acids of the present invention can have one or more functional regions that have an allosteric relationship. For example, a nucleic acid can have an aptamer region and a nucleic acid cleaving region (i.e., DNAzyme region having DNase activity or specific DNA cleaving activity, or a ribozyme region) where the nucleic acid cleaving region is not active when the aptamer region is unbound by ligand. Upon binding of ligand, the nucleic acid undergoes a conformational change such that the nucleic acid cleaving region becomes activated.

In one aspect of the invention, a composition is provided comprising a nucleic acid having at least one aptamer region that specifically binds a ligand and at least one nucleic acid cleaving region. A “cargo molecule,” such as a reporter molecule, an enzyme, or a drug molecule can also be bound or attached to the nucleic acid. The nucleic acid is designed and selected to have an allosteric relationship between the aptamer region and the cleaving region. Binding of a ligand to the aptamer region causes a conformation change such that the nucleic acid cleaving region becomes activated. The activated cleaving region cleaves the nucleic acid in cis and/or nearby nucleic acids in trans resulting in the release of the portion of the nucleic acids that is attached to the cargo molecule. The trans cleavage effect thereby contributes to an amplified response to low levels of ligand by causing the release of multiple cargo molecules in response to a single ligand.

In this manner, if the cargo molecule is a reporter molecule, it is now free to catalyze chromogenic, luminogenic or fluorogenic molecules whereby a colored, chemiluminescent or fluorescent signal is thereby emitted. (The fluorogenic molecules are exposed to light that excites the fluorogenic molecules to fluoresce or substantially increase their fluorescence.) Thus, in one aspect a composition comprises: (a) a nucleic acid comprising (i) an aptamer region that specifically binds a ligand, and (ii) a nucleic acid cleaving cleavage region, and (b) a cargo molecule covalently linked to the nucleic acid, wherein binding of the ligand to the aptamer results in release of the cargo molecule from the nucleic acid. (For example, see FIGS. 1 and 3.)

In the present invention, the cargo molecule can be any molecule that can be attached or bound to a nucleic acid, including, for example, a reporter molecule, an enzyme or a therapeutic drug.

In another aspect, the present invention provides a method for detecting the presence of a ligand involving the steps of (i) contacting a sample with the nucleic acid containing an aptamer region, a cleaving region and an attached cargo molecule (or exposing said nucleic acid to an environment), and (ii) determining whether or not the cargo molecules are released by detecting colored, fluorescent, chemiluminescent, or electric signals. Such detection can therefore involve the nucleic acid composition being bound to a solid surface, such as glass, a bead, a well, a slide, microchip, or a carbon nanotube (CNT) transistor, wherein the solid surface can also be part of a hand-held device. By being bound to a solid surface, the cargo molecule that is attached to the nucleic acid is thereby prevented, prior to ligand binding, from catalyzing a reaction with a chromogenic, fluorogenic or luminogenic molecule or from cleaving a charged molecule, where the chromogenic, fluorogenic, luminogenic or charged molecule is also bound to a surface or CNT transistor. Upon ligand binding and subsequent release of the cargo molecule, its action on the chromogenic, fluorogenic, luminogenic, or charged molecule results in signals that indicate the specific presence of a ligand.

In one aspect, the present invention provides a composition comprising: (a) a well; (b) a nucleic acid comprising (i) an aptamer region that specifically binds a ligand, and (ii) a nucleic acid cleaving region, wherein the nucleic acid is bound to a surface in a first region in the well; (c) a cargo molecule covalently linked to the nucleic acid, wherein binding of the ligand to the aptamer results in release of the cargo molecule from the nucleic acid; and (d) a plurality of fluorogenic molecules bound to a surface in a second region in the well, wherein the surface in the second region comprises a blocking surface, for example, an opaque or metallic surface. (For example, see FIG. 4.) In the present invention, a “blocking surface” is a surface, such as an opaque surface, for example a black painted surface or black plastic, or a metallic surface such as gold, that can prevent the detection of fluorogenic molecules bound to the surface. In this aspect, the bound fluorogenic molecules have a relatively small amount of inherent fluorescence which contributes to detection of non-specific background fluorescence. Reducing the detection of background fluorescence can significantly increase the sensitivity of ligand detection. For example, in another aspect, the blocking surface can block light that would excite the fluorescence of bound fluorogenic molecules. Upon specific binding of a ligand to an aptamer, the binding causes a conformational change in the nucleic acid such that the cleaving region becomes activated (i.e., allosteric effect) and cleaves the nucleic acid such that the cargo molecule is released. The cargo molecule is free to migrate or diffuse to a region in the well (as the constituents in the well can be submerged in a liquid) where fluorogenic molecules are present. Upon excitation by light, the fluorogenic molecules will release fluorescence that can be detected. However, fluorescent signals are not detected without ligand binding because the fluorogenic molecules are tethered to a region in the well that has a blocking surface that locally blocks the exciting light. But if the cargo molecule is an enzyme that can cleave or otherwise release the fluorogenic molecules from the region having the opaque or metallic surface, then the emissions or signals from the released fluorogenic molecules can be detected. In other words, the blocking surface prevents the excitation of the fluorogenic molecules unless these molecules can be released such that they can diffuse away from the blocking surface. Such a composition has multiple levels of signal amplification resulting in great sensitivity in responding to the presence of extremely low levels of ligand.

In the present invention, an enzyme can comprise, for example, an enzyme capable of releasing fluorogenic molecules bound to a blocking surface. The fluorogenic molecules can be derivatized, or otherwise designed to include a peptide region or linker that tethers the fluorogenic molecule to the blocking surface. The enzymes can therefore be selected in view of whether they can selectively cleave the tether.

In another aspect, a composition is provided where release of a cargo molecule is not contingent upon a ligand-aptamer mediated activation of a cleaving domain. For example, a composition can comprise: (a) a well; (b) a first nucleic acid comprising an aptamer region that specifically binds a ligand, wherein the first nucleic acid is bound to the well; (c) a second nucleic acid that is hybridized to the aptamer region (i.e., the second nucleic acid is complementary in sequence to at least a portion of the aptamer region); and (d) a cargo molecule covalently linked to the second nucleic acid; wherein binding of the ligand to the aptamer results in separation of the first and second nucleic acids. (For example, see FIG. 5.) The ligand out-competes the second nucleic acid for an interaction with the aptamer region present in the first nucleic acid resulting in the release of the second nucleic acid. Alternatively, the first and second nucleic acids can hybridize to each other at regions that do or do not involve the first nucleic acid's aptamer domain—in this variation, upon ligand-aptamer binding, the first nucleic acid undergoes a conformational change such that the region that hybridizes to the second nucleic acid is disrupted to the extent that hybridization no longer occurs. Generally, the second nucleic acid can have an attached cargo molecule, such as a reporter molecule that catalyzes the cleavage and activation of chromogenic, luminogenic or fluorogenic molecules. Alternatively, the attached cargo molecule can be an enzyme that cleaves or releases fluorogenic molecules that are tethered to a blocking surface. When the fluorogenic molecules are tethered, the blocking surface prevents the detection of background fluorescence that can be inherent in the fluorogenic molecules, and blocks exciting light from causing fluorescence or from causing the fluorogenic molecules to have increased fluorescence, thereby adding to the accuracy and sensitivity of detection. But upon ligand binding and subsequent release of the second nucleic acid and its bound enzyme, the enzyme is free to diffuse or migrate to the location of the tethered fluorogenic molecules. The free enzyme releases or cleaves the fluorogenic molecules from the blocking surface such that the fluorogenic molecules migrate or diffuse to areas in the well where exciting light is able to cause the fluorogenic molecules to emit fluorescence. The detection of the fluorescence indicates the presence of the ligand. In this aspect, the first and second nucleic acids can each comprise regions of complementary single-strandedness such that they may hybridize to each other.

In another aspect, a composition is provided where an aptamer-ligand interaction does not result in the release of a cargo molecule, but rather in the extension of the nucleic acid's 3-dimensional length such that the cargo molecule has greater reach. For example, a composition can comprise: (a) a well; (b) a nucleic acid comprising a stem-loop structure, wherein the stem comprises an aptamer region that specifically binds a ligand, and wherein the nucleic acid is bound to a surface in a first region in the well; (c) a cargo molecule covalently linked to the nucleic acid; and (d) a plurality of fluorogenic molecules bound to a surface in a second region in the well, wherein the surface in the second region comprises a blocking surface; wherein binding of the ligand to the aptamer results in a dissolution of the stem-loop structure such that the nucleic acid is extended so the cargo molecule reaches in the second region in the well. (For example, see FIG. 6.) In this aspect, ligand-aptamer binding results in a conformational change that disrupts the stem-loop structure (in particular, the stem structure). Because the stem-loop structure is disrupted, the overall reach or extension of the nucleic acid is increased. With the resultant extension, a cargo molecule that is attached or bound to the end of the nucleic acid can now reach a second region in the well. The second region in the well can have fluorogenic molecules tethered to a blocking surface. The cargo molecule, for example, an enzyme that can release or cleave the fluorogenic molecules from the blocking surface will allow the fluorogenic molecules to diffuse or migrate away from the blocking surface. In this manner, detection of fluorescence indicates the specific presence of ligand.

In another aspect, a composition is provided where detection of the presence of a ligand involves a carbon nanotube (CNT) transistor. The CNT transistor allows for even greater sensitivity and accuracy of signal detection. Thus, a composition is provided that comprises: (a) a well (b) a carbon nanotube comprising a transistor, (c) a plurality of charged molecules bound to the exterior of the carbon nanotube, and (d) a nucleic acid comprising (i) an aptamer region that specifically binds a ligand, (ii) a nucleic acid cleaving region and (iii) a cargo molecule covalently linked to the nucleic acid wherein the nucleic acid is bound to the surface of the well, and wherein binding of the ligand to the aptamer results in diffusion of the cargo molecule to the charged molecule. The binding of a ligand to the aptamer is detected by a change in the conductance properties of the CNT, where the change in conductance properties are detected by observing changes in the voltage/current relationship of the CNT transistor. (For example, see FIG. 7.) Ligand-aptamer binding causes a conformational change in the nucleic acid such that the nucleic acid cleaving region becomes active. The nucleic acid cleaving region cleaves the nucleic acid such that the attached cargo molecule is released. The cargo molecule is then able to diffuse or migrate to the location of the well where the CNT is situated. The cargo molecule can then cleave the charged molecules bound to the exterior of the CNT such that this cleavage causes a change in the conductance properties of the CNT. Detection of this change indicates the presence of a ligand.

In another aspect, a composition comprises: (a) a well, (b) a carbon nanotube comprising a transistor, (c) a plurality of charged molecules bound to the exterior of a first region of the carbon nanotube, and (d) a nucleic acid comprising (i) a stem-loop structure, wherein the stem comprises an aptamer region that specifically binds a ligand, and wherein the nucleic acid is bound to the exterior of a second region of the carbon nanotube, and (ii) a cargo molecule covalently linked to the nucleic acid; wherein binding of the ligand to the aptamer results in a dissolution of the stem-loop structure such that the nucleic acid is extended so the cargo molecule reaches into the second region of the carbon nanotube. (For example, see FIG. 11.) In this aspect, ligand-aptamer binding results in a conformational change that disrupts the stem-loop structure (in particular, the stem structure). Because the stem-loop structure is disrupted, the overall reach or extension of the nucleic acid is increased. With the resultant extension, a cargo molecule that is attached or bound to the end of the nucleic acid can now reach the second region of the CNT exterior having the attached charged molecules. The cargo molecule, for example, an enzyme that can release or cleave the charged molecules from the CNT can thereby cause a change in the conductance properties of the CNT, where the change in conductance properties are detected by observing changes in the voltage/current relationship of the CNT transistor. In this manner, detection of the change in conductance indicates the specific presence of ligand.

In another aspect, the invention exploits the chemical sophistication and enzymatic capabilities of nucleic acids combined with the ability of gels to sequester or release interstitial cargo molecules. In this aspect, the invention provides a composition with a nucleic acid comprising an aptamer region that specifically binds a ligand and a nucleic acid cleaving region, wherein the nucleic acid is linked to a matrix subunit thereby forming a matrix, and one or more cargo molecules contained within the matrix. The binding of the ligand to the aptamer results in release of the cargo molecules due to the allosteric relationship(s) possessed by the nucleic acid. The binding of the ligand to the aptamer causes a conformational change in the nucleic acid such that the cleaving region becomes activated. The activated cleaving region cleaves the nucleic acid in cis and/or nucleic acids in trans. As the linkage between the nucleic acids and the matrix subunits is responsible for the integrity of the matrix, the cleavage or fragmentation of the nucleic acids results in the disassembly of the matrix such that the cargo molecules are no longer trapped within the matrix.

Like all aspects of the invention, the above-described ligand-aptamer-mediated gel disassembly aspect can provide numerous applications, such as the detection of environmental ligands or the delivery of drugs. In the application of detection, the cargo molecules can be reporter molecules such as enzymes that catalyze the generation of luminescence or fluorescence of other molecules, such as chromogenic, fluorogenic or luminogenic substrate molecules. Alternatively, the cargo molecule can be an enzyme that can cleave or release fluorogenic molecules bound to a blocking surface. Alternatively, the cargo molecule can be an enzyme that can cleave or release charged molecules bound to a CNT transistor.

A specific ligand can result in both the cis and trans cleavage of nucleic acids that are important to the integrity of the matrix, such that an amplified response is produced because low levels of ligand cause not only a chain reaction of trans cleavage, but also because the disassembly of the matrix can release a large number of reporter molecules. (For example, see FIG. 8.) The response to a ligand is therefore amplified because the response is not limited to a linear relationship between the amounts of ligand and nucleic acid molecules.

Thus, in one aspect, a composition comprises: (a) a nucleic acid comprising (i) an aptamer region that specifically binds a ligand, and (ii) a nucleic acid cleaving region, wherein the nucleic acid is linked to a matrix subunit thereby forming a matrix, and (b) one or more cargo molecules contained within the matrix, wherein binding of the ligand to the aptamer results in release of one or more molecules from the matrix. (For example, see FIGS. 9 and 17.) As stated, the binding of a ligand to an aptamer region can result in the disassembly of a matrix.

In another aspect of the present invention, the ligand-aptamer-mediated gel disassembly design is functionally coupled with different nucleic acids present outside the gel matrix. In this application, the nucleic acids present outside the gel matrix can have an aptamer region that is designed to bind to ligands that are normally too large to pass through the interstitial space of a matrix. Thus, upon binding of a ligand to the aptamer region, the cleavage region becomes activated such that the nucleic acid present outside the matrix self-cleaves and/or cleaves other nucleic acids into fragments. At least one of the resulting fragments is a specific ligand for the aptamer region of the nucleic acids present in the matrix. The binding of the fragment to the aptamer region of the nucleic acid in the gel causes this nucleic acid's cleavage region to become activated, resulting in gel disassembly and release of cargo molecules. (For example, see FIG. 10.)

Thus, in one aspect, a composition comprises: (a) a first nucleic acid comprising (i) a first aptamer region that specifically binds a ligand, and (ii) a first nucleic acid cleaving region, wherein binding of the ligand to the first aptamer region results in cleavage of a fragment from the first nucleic acid; (b) a second nucleic acid comprising (i) a second aptamer region that specifically binds the fragment, and (ii) a second nucleic acid cleaving region, wherein the second nucleic acid is linked to a matrix subunit thereby forming a matrix; and (c) one or more cargo molecules contained within the matrix, wherein binding of the fragment to the second aptamer region results in release of the one or more molecules from the matrix.

In another aspect, the present invention provides a composition with a nucleic acid comprising an aptamer region that specifically binds a ligand, a nucleic acid cleaving region and a terminal hairpin region, where a fluorophore is covalently linked to the hairpin region, and wherein the hairpin structure quenches the fluorescence of the fluorophore. (For example, see FIG. 2.) Upon binding of the ligand to the aptamer, a conformational change results in the nucleic acid such that the cleaving region is activated, resulting in cleavage of the hairpin structure in cis and/or trans. The cleavage of the hairpin prevents the quenching of fluorescence of the fluorophore. In this manner, another method for detecting the presence of a ligand is provided, where if an increase in fluorescence is detected, this indicates the presence of a specific ligand in a sample containing the nucleic acid composition. This aspect is more sensitive than prior methods, because the ligand mediated allosteric control of the nucleic acid cleaving region can result in the cleavage of multiple hairpins, thereby providing a geometric increase in fluorescence from a single ligand-aptamer binding event.

Thus, in one aspect, a composition comprises: (a) a nucleic acid comprising (i) an aptamer region that specifically binds a ligand, (ii) a nucleic acid cleaving region, and (iii) a terminal hairpin region, and (b) a fluorophore covalently linked to the hairpin region, wherein the hairpin structure quenches fluorescence of the fluorophore, wherein binding of the ligand to the aptamer results in cleavage of the hairpin structure, whereby the fluorescence of the fluorophore is no longer quenched.

The present invention also provides aspects where methods for detecting the presence of a ligand comprise: (i) contacting a sample with a composition of the present invention (or exposing the composition to an environment); and (ii) detecting whether or not cargo molecules are released from the matrix, wherein detection of the molecules indicates presence of the ligand in the sample (or in the environment).

In another aspect, methods for detecting the presence of a ligand comprise: (i) contacting a sample with a composition of the present invention (or exposing the composition to an environment), and (ii) detecting whether there is an increase in fluorescence, wherein detection of the increase in fluorescence indicates presence of the ligand in the sample (or in the environment).

In another aspect, methods for detecting the presence of a ligand comprise: (i) contacting a sample with a composition of the present invention (or exposing the composition to an environment), and (ii) detecting whether there is an increase in fluorescence, color, or chemiluminescence from the catalysis of a chromogenic, fluorogenic or luminogenic molecule, wherein detection indicates the presence of the ligand in the sample (or in the environment).

In the application of drug delivery, the aptamer region of the nucleic acid can be designed to bind a physiological ligand such that the gel matrix can release cargo molecules in vivo, where the cargo molecules are drug molecules, thereby providing a controlled drug release application. Further, such an application provides the advantage of releasing predefined amounts of drug cargo molecules in response to low levels of ligand.

In another aspect of the invention, nucleic acids (not necessarily as part of a matrix or gel system) are used to deliver drugs upon responding to physiological stimuli in vivo. The nucleic acids are designed to contain at least one aptamer region that specifically binds to a ligand, a nucleic acid cleavage region, and a region that is coupled or linked to a drug. Upon binding of the ligand, the nucleic acid cleavage region is activated such that the nucleic acid is cleaved, resulting in release of the drug. Alternatively, the nucleic acids can be designed to contain at least two aptamer regions with no cleavage region. In this aspect, one aptamer region is designed to bind to a ligand and the other aptamer region is designed to bind to the drug. Upon binding of the ligand, the nucleic acid undergoes conformational changes such that those aptamer region(s) that bind the drug now release the drug. To modulate the release of drugs with relation to a physiological concentration of a ligand, the nucleic acids can be designed such that more than one aptamer region binds the drug and/or ligand.

Thus, in one aspect, a composition comprises: (a) a nucleic acid comprising (i) a first aptamer region; and (ii) a second aptamer region that specifically binds a ligand; and (b) a drug, wherein the drug is bound to the first aptamer region; and wherein binding of the ligand to the second aptamer region results in release of the drug. Further, in such an aspect, the nucleic acid also comprises a third aptamer region, wherein the drug is bound to both the first and the third aptamer regions in the absence of ligand. In such an aspect, the drug can comprise insulin and the ligand can comprise glucose, for example.

In another aspect, the present invention provides methods for delivering a molecule or a drug within a subject comprising: (a) administering to the subject a composition of the present invention; (b) contacting the composition with a ligand so as to release the molecule or the drug from the composition, thereby delivering the molecule or the drug within the subject. Such methods can be directed to the situation, for example, where the ligand is glucose and the drug or molecule is insulin.

Cargo molecules of the present invention can comprise a reporter molecule, an enzyme, or a therapeutic drug. Reporter molecules of the present invention can catalyze the activation of chromogenic, fluorogenic or luminogenic molecules. Such reporter molecules can be enzymes, which comprise, for example, horseradish peroxidase, alkaline phosphatase, acid phosphatase, β-galactosidase, or β-glucuronidase or a variety of proteolytic enzymes such as subtilisin, trypsin, papain, proteinase K, enterokinase or pepsin.

In the present invention, a chromogenic molecule can comprise, for example, derivatives of 5-bromo-4-chloro-3-indolyl phosphate; 2,2′-azino-di[3-ethyl-benz-thiazoline sulfonic acid; 3,3′,5,5′-tetramethylbenzidine; o-phenylenediamine; p-nitrophenyl-phosphate; o-nitrophenyl-β-D-galactopyranoside; chloro-phenolic red-β-D-galactoopyranoside; or NADP glucose 6-phosphate.

A fluorogenic molecule can comprise essentially any molecule that can fluoresce, for example, a fluorogenic dye. Specific examples of fluorogenic molecules include, for example, derivatives of fluorescein diphosphate; dimethylacridinone phosphate; p-hydroxyphenylacetic acid; 3-(p-hydroxyphenyl) propionic acid; 4-methylumbelliferyl phosphate; 6,8-difluoro-4-methylumbelliferyl phosphate; 4-methylumbelliferyl-β-D-galactopyranoside; fluorescein di-β-D-galactosidase; 4-methylumbelliferyl-galactoside 6-sulfate, GAAAPF-methylaminocoumarin, CAGSGSGPR-7-amino-4-methyl-coumarin or anthraniloyl-Lys-p-nitroanilide. Fluorogenic molecules can also include derivatized fluorogenic molecules. Derivatized fluorogenic molecules can be, for example, fluorogenic molecules that have attached immobilizing groups that tether the molecule to a solid surface. The derivatized fluorogenic molecule can also include, either as part of the immobilizing group or as a region between the immobilizing group and the fluorogenic part of the molecule, a region that can be specifically cleaved, either by a protein enzyme or by a nucleic acid cleaving region. The derivatized fluorogenic molecule can also be a fluorogenic peptide, where the peptide includes a region that can be specifically cleaved by a protein enzyme (without harming the fluorogenic potential of the peptide) and an immobilizing region that tethers the peptide to a solid surface. Examples of groups that can be used to immobilize a peptide include Cys residues to attach a peptide to a gold surface, and Lys residues that attach the peptide to an aldehyde activated surface.

A luminogenic molecule can comprise, for example, derivatives of 1,2-dioxetanes; luminol; coelenterazines; luciferins; acridines; or metal ions.

Such chromogenic, fluorogenic or luminogenic molecules can be attached to a surface or a solid support.

In the present invention, an enzyme can comprise, for example, an enzyme capable of releasing or cleaving a charged molecule. Examples of enzymes include subtilisin, hyaluronidase, chitinase, cellulase, phospholipase C, trypsin or DNA restriction enzymes. A charged molecule can be negatively charged or positively charged and can be peptides, nucleic acid polymers or lipids. Examples of charged molecules include peptides with a subtilisin cleavage site, hyaluronic acid, chitosan, carboxymethylcellulose, dipalmitoyl-phosphatidyl-inositol-diphosphate, or double stranded DNA.

In the present invention, the nucleic acids can comprise DNA or RNA. Further, the nucleic acid cleaving regions can comprise a ribozyme or a DNAzyme. The nucleic acids of the present invention can further comprise a recognition region recognized by the nucleic acid cleaving region. Further, an aptamer region can comprise from about 15 to about 500 nucleotides, from about 15 to about 200 nucleotides, from about 15 to about 100 nucleotides, or from about 40 to about 200 nucleotides, for example. Further, a nucleic-acid cleaving region can comprise from about 15 to about 500 nucleotides, or from about 15 to about 200 nucleotides, from about 15 to about 100 nucleotides, or from about 40 to about 200 nucleotides, for example.

Ligands contemplated by the present invention can comprise, for example, one or more of an ion, a small organic molecule, nucleic acids, proteins, viruses, fungi, bacteria cells, chemical toxins, bioterrorism agents, pollutants, allergens, irritants, physiological indicators or any combination thereof. A physiological indicator can comprise, for example, glucose, calcium, uric acid, cholesterol, vitamin D, creatinine, bilirubin, triglycerides, hormones, or any combination thereof. Specific non-limiting examples of ligands relating to bioterrorism include anthrax spores, ricin, botulotoxin, nerve gases, trinitrotoluene, dioxin, small pox, and plague.

In the present invention, a matrix subunit can comprise, for example, one or more of polyacrylamide, polysaccharide, polystyrene, polypropylene, polyethylene, polyurethane, polysiloxane, polymethyl methacrylate, polyvinyl alcohol, polyethylene, polyvinyl pyrrolidone, or any combination thereof.

### BRIEF DESCRIPTION OF THE DRAWINGS

FIG. 1 depicts the effect of ligand (“L”) binding to a nucleic acid (“X”) having an aptamer region (“A”), a nucleic acid cleaving region (“C”) and an attached cargo molecule (“CM”). In step 1, nucleic acid X is unbound with ligand. In step 2, the ligand binds to nucleic acid X's aptamer region, causing a conformational change whereby the nucleic acid cleaving region becomes activated. The active cleaving region fragments the nucleic acid X (cis-cleavage) and nearby nucleic acids, such as nucleic acid Y (trans-cleavage), resulting in the release of cargo molecules (step 3). Not depicted in this figure is the possibility that the cleaving region fragments the nucleic acid upstream of the cleavage region (the nucleic acid can be designed such that the cleaving region can also specifically fragment the nucleic acid either upstream or downstream of the cleaving region itself). In this scenario, the activated cleaving region is not limited to cleaving only nearby nucleic acids, thus the amplified response to ligand can entail cleavage of both nearby and non-adjacent nucleic acids in response to a single ligand-aptamer interaction. Further, instead of a CM representing a cargo molecule, the CM can also represent a matrix subunit (additionally, both ends of the nucleic acid can be attached to matrix subunits). In this situation, the activated cleaving region results in the fragmentation of nucleic acids that crosslink matrix subunits in cis and in trans.

FIG. 2 depicts an embodiment where the nucleic acids comprise an aptamer region (“A”), a nucleic acid cleaving region (“C”), a hairpin region (“H”) and an attached fluorophore or fluorescent adduct (“F”). In step 1, nucleic acid X is unbound with ligand, and the fluorophore's fluorescence is quenched by the hairpin region. In step 2, the ligand (“L”) specifically binds to the aptamer region causing a conformation change such that the cleaving region is now active. The cleaving region cuts the hairpin region in cis and in trans, resulting in release of the fluorescent adduct (alternatively, the cleavage can simply disrupt the hairpin structure without cleaving the structure from the nucleic acid) such that the fluorophore is no longer quenched. As stated above in FIG. 1, the cleaving region can be designed such that it can cleave the nucleic acids either upstream or downstream of the cleaving region, enabling trans cleavage of non-adjacent nucleic acids.

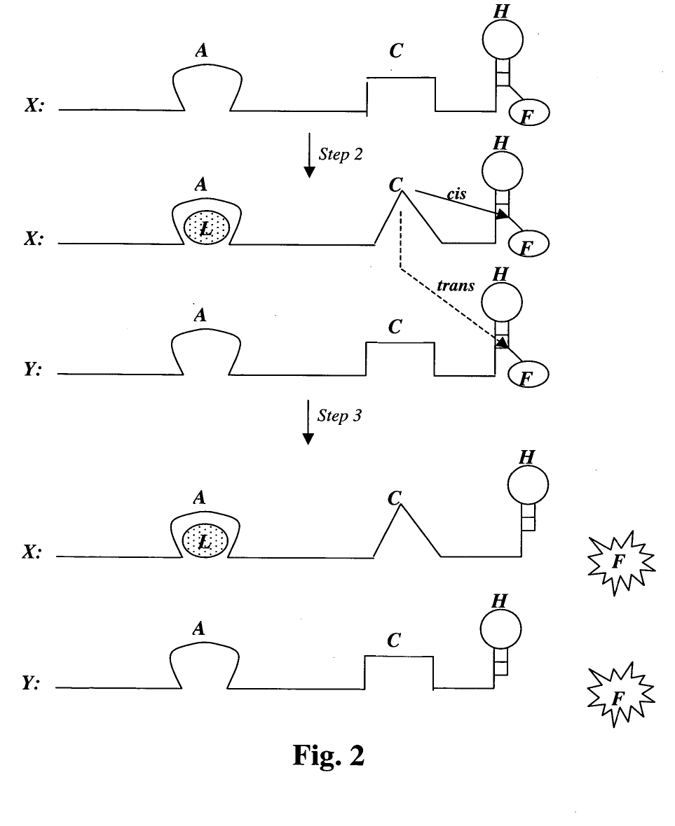

FIG. 3 depicts the release of a cargo molecule by cleaving a DNA tether with a DNAzyme. A cargo molecule (e.g., the enzyme alkaline phosphatase) can be attached to a solid surface using an allosteric DNAzyme as a tether. When the aptamer comes to contact with its ligand, the DNAzyme will be switched on, the tether will be cleaved, and the cargo molecule is free to migrate to the nearby area of the surface populated with attached substrate molecules. An example of a method to attach the substrate molecules to the surface is by conjugating the substrate molecules to biotin then applying the biotin-conjugated substrate molecules to avidin-coated solid surfaces. In this example, the substrate will be a fluorogenic molecule, for example, a phosphorylated fluorogenic dye such as marina blue phosphate. When the phosphate moiety is cleaved from the fluorogenic dye, its fluorescence sharply increases. Thus the specific interaction between a ligand and an aptamer is detected as an increase in fluorescence.

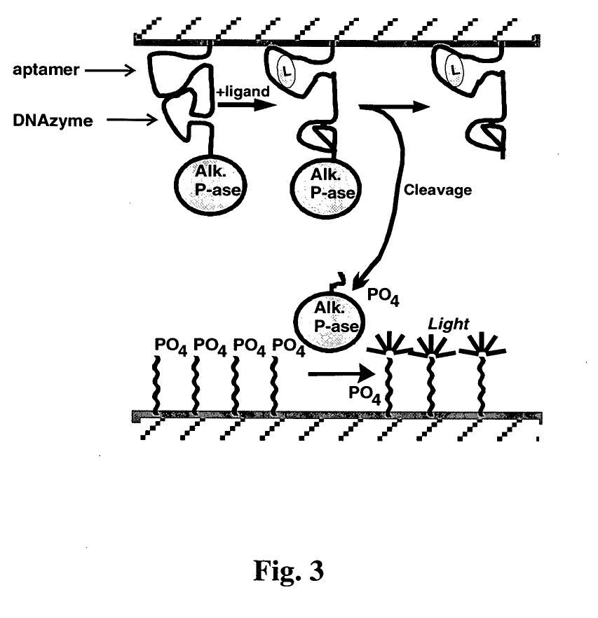

FIG. 4 depicts a method for improving detection sensitivity by separation of a fluorescent product from the solid surface to which it is attached. Since the substrate is necessarily immobilized, then a separation of the fluorescent product can be effected by simply letting it diffuse away from the site of the immobilized substrate. Peptide substrates are cleaved by proteases and can be derivatized by adding an immobilization group to one end and a fluorogenic dye to the other end. When a protease cargo molecule is released after its DNAzyme tether is cleaved, the protease cleaves the fluorogenic peptide, and the fluorescent product of this cleavage exhibits greatly increased fluorescence and at the same time becomes free to diffuse in solution. In this case the fluorogenic peptide would be immobilized on an opaque surface such as a gold disc or gold bead to shield the substrate molecule from the exciting illumination thus giving no rise in fluorescence or background fluorescence. Once cleaved, the fluorescent product diffuses away from the opaque barrier into a region that is illuminated and therefore displays its full fluorescence. There are many fluorogenic peptides that are suitable for being bound to blocking surfaces. Peptides that can be cleaved by enzymes such as proteases can be derivatized by adding an immobilization group to one end and a fluorogenic molecule or dye to the other end.

FIG. 5 depicts the release of a cargo molecule that is tethered to a solid surface by DNA hybridization. This method, called “hybridization competition”, improves specificity controlling the release of the cargo molecule and is based on the ability of ligand binding to cause a DNA aptamer to switch conformations. A cargo molecule (depicted here as a protease “Pr”) can be attached to a solid surface via base pairing between the two strands of a double-stranded DNA molecule. The strand of DNA that is attached to the cargo molecule is complementary to the strand of DNA that is bound to the substrate or surface. The DNA strand that is attached to the surface would be comprised of an aptamer. Upon presentation with ligand (“L”), the ligand would compete against the strand of DNA conjugated to the cargo molecule for interaction with the aptamer strand causing release of the cargo molecule. The length and number of base pair density of the two DNA strands would be adjusted such that there is no release of the cargo molecule in the absence of ligand, but some release when ligand is present.

FIG. 6 depicts a stem-loop DNA structure to tether the cargo molecule to a solid surface. This is a variation of the method in FIG. 5. In this configuration the cargo molecule (“Pr”, protease) remains tethered to the solid surface while undergoing a reaction with substrate molecules (“F”, fluorogenic substrate molecule). The length of the tether changes upon contact with the aptamer. The tether is comprised of a relatively long single stranded DNA molecule that contains an aptamer region near the end that is attached to the solid surface and a complementary sequence near the other end, which is attached to the cargo molecule. In the absence of ligand, these two regions are free to hybridize and the DNA takes on a stem-loop shape with a long loop connecting the aptamer-anti-aptamer double stranded stem. Since both ends are together at the base of the stem, the cargo molecule is constrained to an area close to the point of attachment. Upon binding of the ligand (“L”), the aptamer would adopt a new conformation that would preclude its participation in the aptamer-anti-aptamer double stranded stem. With no stem, the tether would realize its full length, allowing the cargo molecule to reach the substrate molecules.

FIG. 7 depicts a sensor that uses a carbon nanotube (“CNT”) to generate an electrical signal in response to conformational changes induced by ligand-aptamer binding (see Example 1). The present invention uses an enzyme cargo molecule, for example a protease, as a carbon nanotube modifying agent. The protease released from the aptamer tether modifies the carbon nanotube environment by cleaving the carbon nanotube modifier attached to the surface of the carbon nanotube, here depicted as a negatively-charged peptide, and thus producing a detectable change in the field effect transistor behavior of the carbon nanotube. The nucleic acid comprising the aptamer and protease is bound to a solid surface adjacent to a short carbon nanotube field effect transistor connected to gold terminals and embedded in a plastic medium compatible with exposure to aqueous environments. The carbon nanotube is decorated with highly negatively-charged peptide molecules. The released protease will diffuse to the nearby carbon nanotube and cleave the peptides. As the charged peptides are released from the carbon nanotube, the conductance properties of the carbon nanotube will change and are detected as a change in the voltage/current relationship of the field effect transistor.

FIG. 8 depicts the disassembly of a matrix upon ligand-aptamer binding. The spheres represent cargo molecules located in the interstitial spaces of the matrix. Upon disassembly, the cargo molecules are released (top arrow). In the bottom situation, a single nucleic acid of the matrix is depicted. The left-hand side represents the unbound state of the nucleic acid, where the nucleic acid crosslinks matrix subunits on either end. After ligand binding, the cleaving region enacts cis-cleavage such that the nucleic acid no longer forms a link between two matrix subunits.

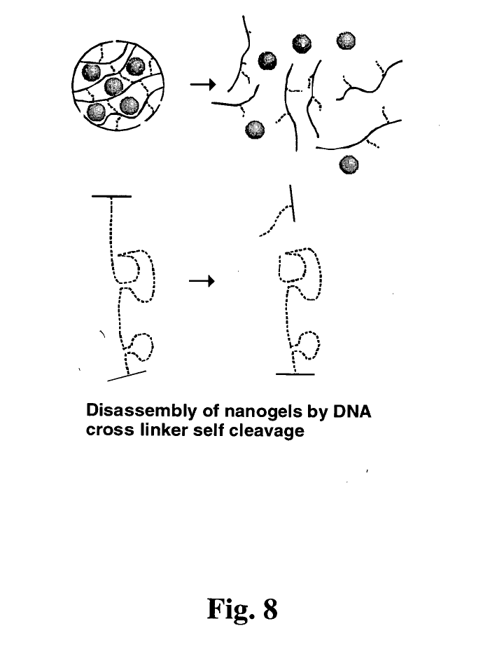

FIG. 9 depicts an embodiment of matrix-mediated detection. The spherical object in the top panel represents the matrix. In this example, the cargo molecules contained within the matrix is a reporter molecule, bacterial alkaline phosphatase. The matrix also comprises nucleic acid molecules that cross-link matrix subunits into a three-dimensional matrix. The nucleic acid molecules comprise an aptamer domain that specifically binds adenosine, and a nucleic acid cleaving region (deoxyribozyme DNase). The matrix is present in a well filled with solution, where fluorogenic substrate molecules, fluorescein phosphate, are bound to the surface of the well. This attachment of the fluorescein phosphate molecules is to prevent their interaction with the alkaline phosphatase in the matrix prior to ligand binding. When the adenosine (“A”) ligand enters the well, it binds to the aptamer region of the nucleic acid. This binding causes a conformational change in the nucleic acid such that cleaving region becomes activated. The cleaving region amplifies the response to the ligand by cleaving the nucleic acid in cis and other nucleic acids in trans. The fragmented nucleic acids no longer cross-link the matrix subunits to each other, causing a disassembly of the matrix. The disassembly of the matrix further amplifies the response to ligand as the disassembled matrix can release a great number of cargo molecules in response to limited ligand-aptamer binding. When the alkaline phosphatase is released from the matrix, it is free to dephosphorylate the fluorescein phosphate substrate, catalyzing the fluorescence of the substrate. Detection of the fluorescence therefore indicates the presence of the ligand.

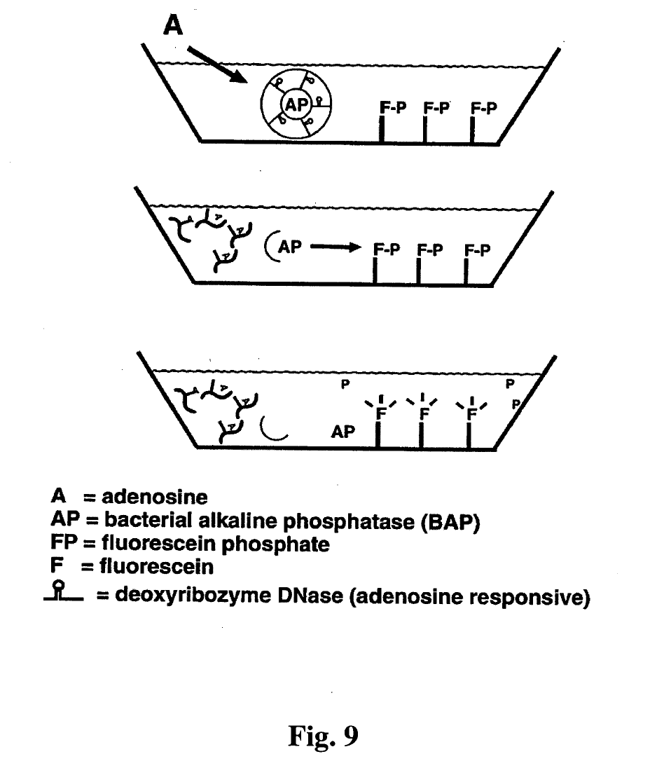

FIG. 10 depicts an embodiment of detection where a ligand is too large to enter the interstitial spaces of the matrix. In this figure, nucleic acids that are present outside the matrix (the nucleic acids with TCCC at the 3′ end) comprise an aptamer region that specifically binds thrombin (the ligand) and a nucleic acid cleaving region (deoxyribozyme DNase). Upon binding of thrombin to the aptamer region, the nucleic acid undergoes a conformational change such that the cleaving region becomes active (“thrombin responsive”). The cleaving region fragments the nucleic acid in cis and/or in trans, where the cleaving region specifically fragments the nucleic acid such that a TCCC fragment is created. The TCCC fragment specifically binds aptamer regions of the nucleic acids present in the matrix. Upon binding of the TCCC fragment, the nucleic acids in the matrix undergo a conformational change where their cleaving regions are thereby activated. These cleaving regions fragment the matrix nucleic acids causing disassembly of the matrix and release of the alkaline phosphatase cargo molecules. The alkaline phosphatase dephosphorylates the immobilized fluorescein phosphate resulting in fluorescence that can be detected by a variety of methods known in the art.

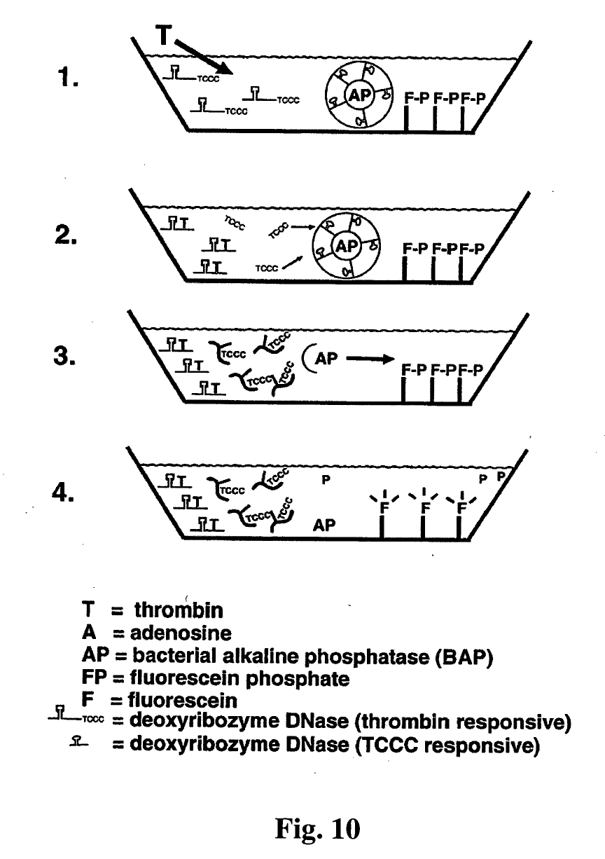

FIG. 11 depicts a variation of the carbon nanotube sensor in FIG. 7 comprising the use of a tether extension (as in FIG. 6) to release the protease cargo molecule rather than cleavage of the tether (see Example 1). The protease cargo molecule (“P”) is tethered to a carbon nanotube by an aptamer that forms the stem in a stem-loop DNA structure. In the presence of ligand (“L”), the aptamer would be de-hybridize from its complementary strand, binding the ligand as a mutually exclusive alternative. The DNA, now in a linear conformation, would extend, allowing the cargo molecule to extend over a long distance to reach charged molecules bound to the carbon nanotube.

FIG. 12 depicts a nucleic acid that contains an allosteric relationship between its aptamer region and its DNAzyme region (see Example 2). The aptamer region modulates the DNAzyme region because the DNAzyme only cleaves in the presence of glucose.

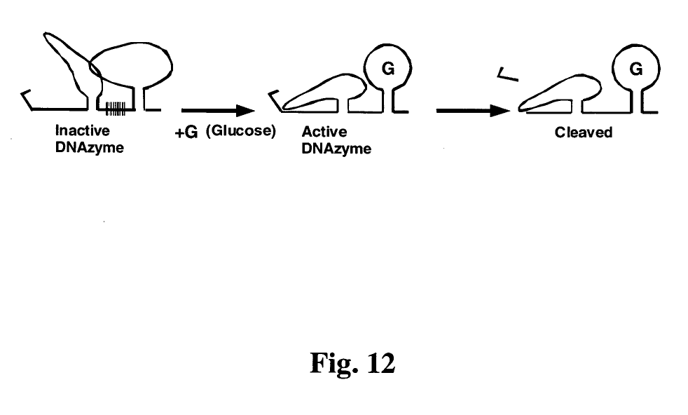

FIG. 13 depicts a nucleic acid that provides the controlled release of a cargo molecule through the use of multiple aptamer domains (see Example 4). In the figure, the nucleic acid molecule contains two aptamer domains that bind insulin (“I”) when the aptamer that specifically binds to glucose is unbound. The binding of glucose to the aptamer domain causes a conformational change in the nucleic acid such that the insulin molecule is released.

FIG. 14 depicts a selection scheme for a nucleic acid having a cleaving region that is activated by glucose. The selection scheme is described in Example 2.

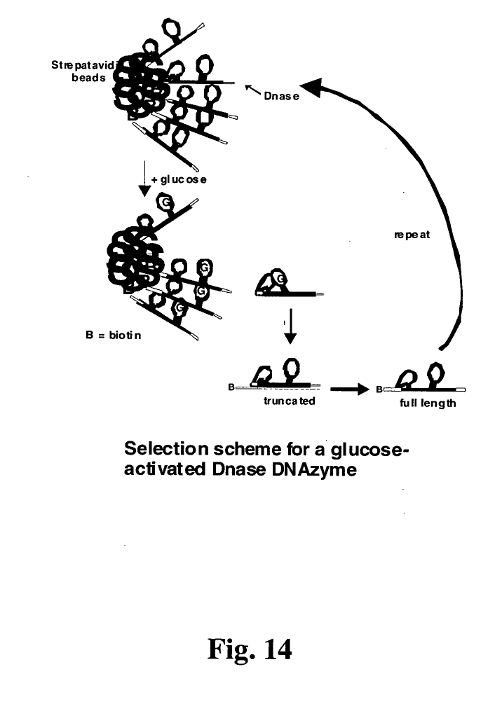

FIG. 15 depicts different types of reactions that can be used in the synthesis of matrices (see Example 5).

FIG. 16 depicts two types of matrices, layered Nanogels and core shell nanogels (see Example 5).

FIG. 17 depicts the disassembly of nanogels and release of a protein enzyme (shown here as alkaline phosphatase) by ligand-activated DNA cross-linker self-cleavage. The protein enzyme would be trapped in small polyacrylamide gel spheres (nanogels) 50 to 500 nm in diameter. The porosity of the gel would be such as to preclude significant leakage of the enzyme. The gel would be formed using DNA to crosslink the polyacrylamide strands. This DNA would be comprised of an allosteric DNAzyme or by a DNA aptamer hybridized to its complementary sequence. When a small molecule target ligand is present, it would diffuse into the nanogel, activate the DNAzyme, which would break the crosslink, disassembling the gel and releasing the protein enzyme. The last would diffuse to the region of solid-state fluorogenic substrate, cleave the fluorogenic moiety and produce the fluorescent signal.

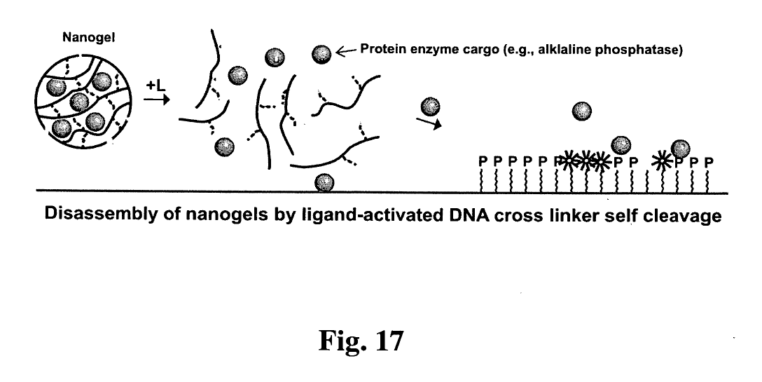

FIGS. 18A-C depict the following aptamer-based biosensor schemes: a beacon aptamer (FIG. 18A), an aptazyme sensor (FIG. 18B) and a tethered protein enzyme sensor (FIG. 18C) (see Example 7).

FIG. 19 depicts a model amplified-based biosensor for copper as described in Example 7.

FIG. 20 depicts DNAzyme self-cleavage in the absence and presence of copper only, or copper and ascorbate (see Example 7).

FIG. 21 depicts self-cleavage of the DNAzyme attached to an avidin bead (see Example 7).

FIGS. 22A-B depict conjugation of the DNAzyme to trypsin as described in Example 7. FIG. 22A depicts a Coomassie blue stain for protein. FIG. 22B depicts a SYBR gold stain for nucleic acid.

FIG. 23 depicts copper-dependent cleavage of the trypsin-DNAzyme conjugate as described in Example 7. The left panel is an SDS-PAGE gel with Coomassie blue staining for protein. The right panel is a urea/TBE-PAGE gel with SYBR staining for DNA.

FIG. 24 depicts release of the tethered trypsin by copper (see Example 7).

FIG. 25 depicts the emission spectrum of the fluorescent material (coumarin) released by copper exposure (see Example 7).

FIG. 26 depicts the detection of increasing concentrations of copper using the fluorescence readout of the allosteric aptazyme-based biosensor (see Example 7).

### DETAILED DESCRIPTION OF THE INVENTION

The present invention is directed to the use of nucleic acid functionalities in order to detect and respond to ligands in external and physiological environments. Further, the present invention uses the ability of nucleic acids to detect molecular and macromolecular ligands in order to provide drug delivery methods and devices that are sensitive to physiological or man-made conditions.

The present invention takes advantage of the ability of nucleic acids to have multiple functional regions on the same molecule, where these regions may possess an allosteric relationship between each other. In the present invention, an allosteric relationship is present if a nucleic acid has at least two functional regions, one of which is an aptamer region, where the binding of a specific ligand to the aptamer region causes a conformational change such that another functional region is affected. For example, in certain embodiments, the present invention provides nucleic acids that contain an aptamer region and a nucleic acid cleaving region. By “nucleic acid cleaving region,” it is meant that a stretch of the nucleic acid is itself a functional domain, for example, having the ability to cleave or fragment nucleic acids in cis and/or in trans. The cleavage can occur at a specific target sequence, depending upon the design of the nucleic acid cleaving region (for example, a ribozyme or DNAzyme can be created such that they cleave only at a specific nucleotide recognition sequence). However, the cleaving region is inactive until the nucleic acid undergoes the ligand-aptamer dependent conformational change.

The present invention provides the advantage of being able to respond to a ligand in an amplified manner, in part because an active nucleic acid cleaving region can fragment nucleic acids in cis and in trans. Although the scope of the present invention does contemplate the design of multiple different aptamer regions that can bind to a single complex ligand, such as a protein (thereby allowing multiple nucleic acid molecules to bind to a single ligand at a given moment), the present invention also seeks to amplify the response to a ligand-aptamer(s) event mainly through the use of allosteric relationships between nucleic acid functional regions, such as between two or more aptamer regions or between an aptamer region(s) and a nucleic acid cleaving region(s), etc. Additional levels of signal amplification are provided at least through the use of: (1) enzyme cargo molecules that can cleave or release fluorogenic molecules tethered to blocking surfaces; (2) gel disassembly compositions that cleave release numerous cargo molecules in response to low levels of ligand; (3) blocking surfaces that reduce or eliminate the detection of background fluorescence; and (4) CNT transistors that provide extremely accurate and sensitive methods for detecting the presence of ligands.

In one embodiment, nucleic acids have at least one aptamer region, a nucleic acid cleaving region, a hairpin region and a bound fluorophore, where the hairpin region quenches the fluorescence of the fluorophore. Upon binding of the specific ligand to the aptamer region, a conformational change causes the nucleic acid cleaving region to become active. The cleaving region then fragments or cleaves nucleic acids in cis and in trans, resulting in the dequenching of multiple fluorophores in response to one ligand-aptamer event.

In another embodiment, nucleic acids are provided that have at least one aptamer region, a nucleic acid cleaving region and a cargo molecule that is bound or linked to the nucleic acid. Binding of a specific ligand to the aptamer region causes a conformational change in the nucleic acid such that the cleaving region is now active. The active cleaving region not only can cleave the same nucleic acid molecule on which it resides, i.e., cis cleavage, but the active cleaving region can cleave other nucleic acid molecules, i.e., trans cleavage. The result of the cleavage will be the release of the cargo molecule from the nucleic acid. In the application of detection, the cargo molecules can be reporter molecules such as enzymes that can catalyze reactions with fluorogenic, luminogenic or chromogenic molecules. Further, the nucleic acids can be attached to a solid surface, such as a well, slide or a microchip array. The attachment of the nucleic acid to the solid surface prevents the bound reporter molecules from reacting with substrates prior to a ligand-aptamer interaction. In other words, by attaching the nucleic acids to a surface, the bound reporter molecules are prevented from creating fluorescent, luminescent or chromogenic signals that are not ultimately dependent upon specific ligand-aptamer interaction. In addition, such a detection method contemplates that the nucleic acids and the reporter molecule substrates are bound to a solid-surface that holds a solution. The solution enables the unbound reporter molecules to travel to the region of the surface where the fluorogenic, luminogenic, or chromogenic substrate molecules are located.

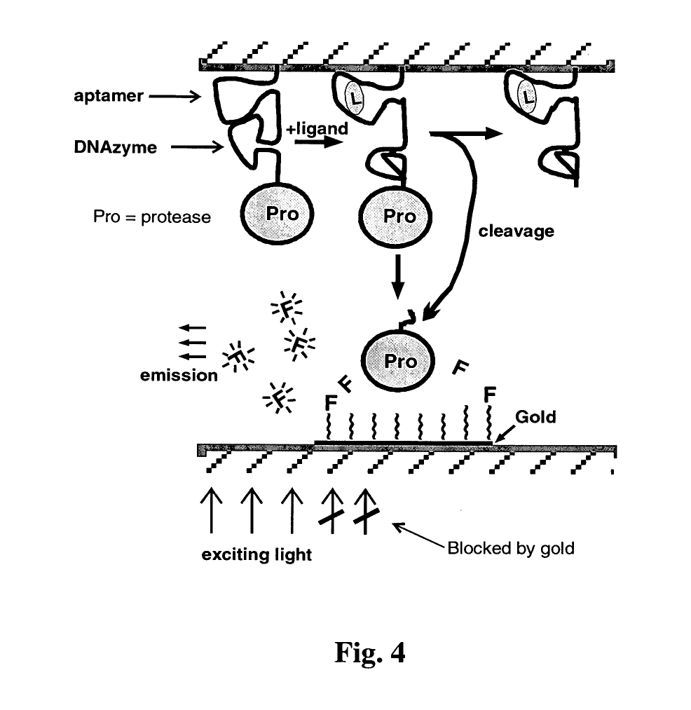

In one embodiment, a nucleic acid comprising an aptamer region, an allosteric DNAzyme region, and a bound protein enzyme (such as a protease) is attached to the surface of a well. When the aptamer comes into contact with its ligand, the DNAzyme will be switched-on and the nucleic acid will be cleaved such that the protein enzyme is released. The protein enzyme is free to migrate to an area of the well that is populated with substrate molecules, such as fluorogenic molecules, attached to the surface of the well. The substrate molecules can be fluorogenic, chromogenic or luminogenic molecules that when cleaved by the protein enzyme, the fluorescence or chemiluminescence of the substrate molecules sharply increases. Therefore, one molecule of ligand can bring about the cleavage of many tethers and thus release many molecules of protein enzyme, each of which can transform thousands of substrate molecules into thousands of fluorescent signals. The two levels of amplification intrinsic to this scheme should allow detection of a target with unprecedented sensitivity in a miniature spot on a slide or chip. In relation to this embodiment, sensitivity can be further improved by immobilizing a fluorogenic molecule, such as a fluorogenic peptide, on a blocking surface. The exciting light will not penetrate the blocking surface and any potential inherent background fluorescence from the fluorogenic molecule would thus be shielded from a detector, such that no detectable signal will be contributed by the uncleaved fluorogenic substrate molecule. Once cleaved by an enzyme, the fluorogenic molecule will diffuse away from the blocking surface into a region that is illuminated by exciting light and therefore display its full emission of fluorescence. (For example, see FIG. 4).

Another aspect of the invention encompasses a nucleic acid comprising an aptamer region, an allosteric DNAzyme region, and a bound fluorogenic molecule is attached to the blocking surface of a well. When the aptamer comes into contact with its ligand, the DNAzyme will be switched-on and the nucleic acid will be cleaved such that the fluorogenic molecule is released. Once the nucleic acid is cleaved, the fluorogenic molecule will diffuse away from the blocking surface into a region that is illuminated by exciting light and therefore display its full emission of fluorescence.

Embodiments using fluorogenic molecules tethered to a blocking surface improves the signal to noise ratio of detection. In certain embodiments, the cargo molecule is a reporter enzyme that can cleave a fluorogenic molecule itself, where the cleavage results in increased fluorescence of the fluorogenic molecule. For example, alkaline phosphatase will cleave phosphate moieties off of phosphorylated fluorogenic dyes; when the phosphate moiety is cleaved, the fluorescence sharly increases. Examples of such phosphorylated fluorogenic dyes are pacifica blue phosphate (ethylenediamine pacifica blue phosphate), marina blue phosphate and 6,8-difluoro-4-methylumbelliferyl phosphate. Although cleavage of the phosphate from certain fluorogenic substrates result in a large increase in fluorescence, the fluorescence of the uncleaved phosphorylated fluorogenic substrate can contribute background fluorescence. Thus, as mentioned, to eliminate the background fluorescence, the present invention provides compositions that tether fluorogenic substrates to blocking surfaces. The fluorogenic molecules can be tethered to the blocking surfaces by derivatizing peptides to comprise an immobilization group on one end of the peptide and a fluorogenic dye on the other end of the peptide. Examples of peptide immobilization groups include Cys residues to attach peptides to a gold surface, and Lys residues to attach peptides to an aldehyde activated surface. The fluorogenic peptides are designed such that enzymes, such as proteases, will specifically cleave the peptide such that the fluorogenic dye is free to migrate or diffuse away from the blocking surface.

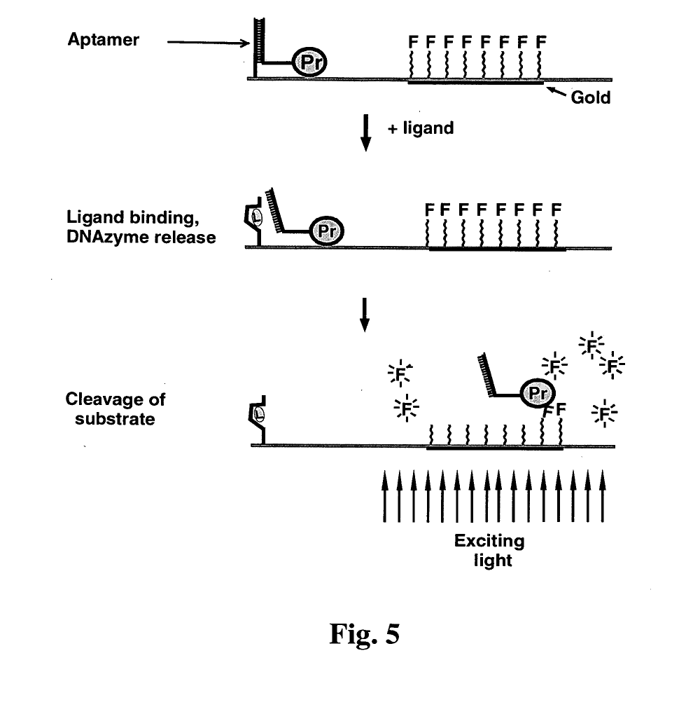

Further aspects of the invention additionally provide for controlling the release of a protein enzyme cargo molecule. The methods are based on the ability of a ligand to cause a change in the conformation of the aptamer. For example, the release of the protein enzyme can be controlled by “hybridization competition” between the immobilized aptamer and its ligand or a complementary strand of DNA attached to the cargo molecule. A protein enzyme would be tethered to a solid support via base pairing between two strands of a double-stranded DNA molecule. The strand of DNA that is conjugated to the protein would be complementary to a strand of DNA that is bound to a solid surface. The DNA strand that is attached to the solid surface would be comprised of an aptamer. Upon presentation with ligand, the ligand would compete against the strand of DNA conjugated to the protein enzyme for interaction with the aptamer strand (Nutiu, R. and Li, Y., Chemistry, 2004, 10:1868-1876). The length and number of base pair density of the two DNA strands would be adjusted such that there is no release of the protein enzyme in the absence of ligand, but some release when ligand is present. Once the protein enzyme is released it would diffuse away, making the release essentially irreversible (see FIG. 5). The protein enzyme could be trypsin used in conjunction with a fluorogenic peptide such as anthraniloyl-Lys-p-nitroanilide that is tethered to a blocking surface. The released protein enzyme will specifically cleave the fluorogenic peptide at its immobilization group, thereby releasing the portion of the peptide with the fluorogenic dye. The released fluorogenic dye will be free from the blocking surface that blocks or absorbs exciting light, such that the exciting light will cause the dye to release its full fluorescence.

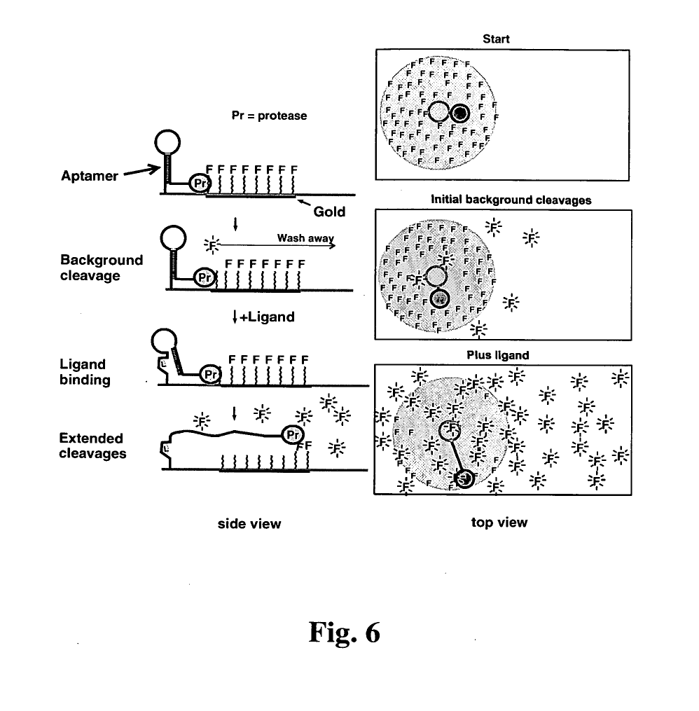

The invention also provides for a variation on the hybridization competition scheme to further control the diffusion of the protein enzyme cargo molecule. In this configuration, the protein enzyme is never released to freely diffuse to the location of the substrate molecules, but rather it remains tethered to a solid surface. What changes upon contact with the aptamer is the length of the tether. The tether here is comprised of a relatively long single stranded DNA molecule that contains an aptamer region near the end that is attached to the solid surface and a complementary sequence near the other end, which is attached to the protein enzyme. In the absence of ligand, these two regions are free to hybridize and the molecule would take on a stem-loop shape with a long loop connecting the aptamer-anti-aptamer double stranded handle. The sequence of the loop would be designed to minimize any folded structures (e.g., it could consist exclusively of thymine bases). For example, the geometry of the stem-loop structure can be a stem of 30 base pairs supporting a loop of 65 thymine nucleotides and a short initial tether of 15 nucleotides. Since both ends are together at the base of the stem, the protein enzyme will be constrained to an area close to the point of attachment. Surrounding each solitary attached DNA-protein complex would be thousands of solid state substrate molecules. When these components are first assembled, the protein enzyme would cleave those few substrate molecules it could reach on this short constrained tether; these immediate products can be washed away. Upon subsequent binding of the ligand, the aptamer would adopt a new conformation that would preclude its binding to the anti-aptamer sequence. With no stem, the tether would realize its full length, allowing the protein enzyme to reach hundreds of times more substrate molecules (see FIG. 6).

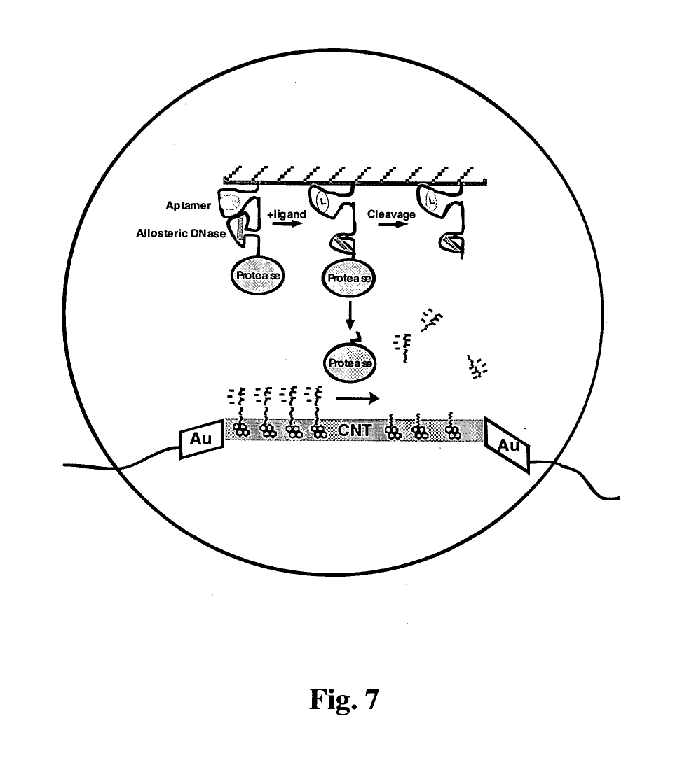

The present invention provides for the detection of ligand-aptamer binding by transducing the interaction directly into an electrical signal using carbon nanotubes. For example, when connected to gold terminals and subjected to voltage sources, the nanotube acts as a semiconductor that can be configured as a field effect transistor. The conductance of the carbon nanotube field effect transistors is sensitive to their immediate chemical or ionic environment (Someya, T. et al., Appl. Phys. Lett., 2003, 82:2338-2340; Chen, R. J. et al., J. Am. Chem. Soc., 126:1563-1568; Someya T. et al., Nano. Letters, 2003, 3:877-881). The ionic environment of the carbon nanotube can be altered by binding or removing charged molecules to or from the carbon nanotube. Charged molecules can be negatively charged or positively charged and can be peptides, nucleic acid polymers, polysaccharides or lipids. For example, as charged peptides attached to the carbon nanotube are released due to cleavage of the peptides by protease cargo molecules, the conductance properties of the carbon nanotube will change, detectable as a change in the voltage/current relationship of the field effect transistor. In one embodiment, signal amplification can be effected by using an allosteric self-cleaving DNAzyme to tether a protein enzyme molecule (such as a protease) to a solid surface in a small well. Each time a ligand binds to the aptamer region of the tether, it will self-cleave, releasing the protein enzyme. Also present in the well adjacent to the aptamer-protease can be a short carbon nanotube connected to gold terminals and embedded in a plastic medium compatible with exposure to aqueous environments (Someya, T. et al., Nano. Letters, 2003, 3:877-881). The carbon nanotube can be decorated with highly negatively charged peptide molecules. The released protease will diffuse to the nearby carbon nanotube and cleave the bound peptides. As the charged peptides are released from the carbon nanotube, its conductance properties will change, detectable as a change in the voltage/current relationship of the field effect transistor (Someya, T. et al., Nano. Letters, 2003, 3:877-881; Chen, R. J. et al., J. Am. Chem. Soc, 2004, 126:1563-1568). A single protease molecule should suffice to cleave all of the several hundred peptide molecules from a single short (100 nanometers) carbon nanotube, providing an amplified response to the presence of a ligand, where the method of detecting changes in conductance through the CNT transistor provides extreme sensitivity, accuracy and applicability with high-throughput and miniaturized devices. This scheme is depicted in FIG. 7. It should be noted that in this system the signaling molecules accumulate over time, allowing signal integration.

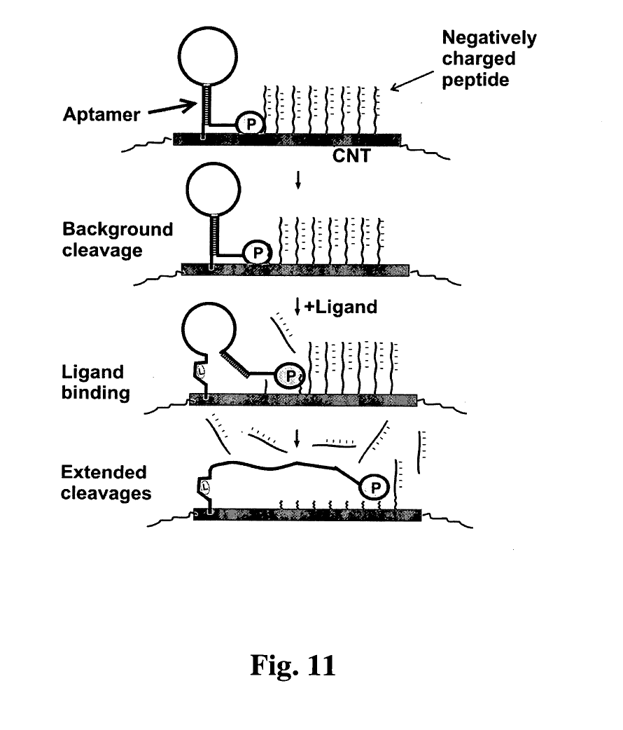

In a variation on this theme, the protease would be tethered to a carbon nanotube by an aptamer that forms the stem in a stem-loop DNA structure; this molecule would be surrounded by hundreds of substrate molecules on a 10 micron carbon nanotube. In the presence of ligand, the aptamer would be de-hybridize from its complementary strand, binding the ligand as a mutually exclusive alternative (Nutiu, R. and Li, Y., Chemistry, 2004, 10:1868-1876). The DNA, now in a linear conformation, would extend, allowing the tethered protease to claeave substrates over a long distance on the carbon nanotube (see FIG. 11).

In other embodiments, nucleic acids comprising an aptamer region, a nucleic acid cleaving region and a bound cargo molecule can also constitute a drug delivery method or device. In this aspect, the cargo molecule comprises a drug, such as a therapeutic small molecule or a therapeutic protein. Such nucleic acid molecules can be attached to biocompatible polymers, for example those described in U.S. patent application publications U.S. 2003/0008818 and U.S. 2003/0017972, whereby the polymers facilitate the delivery of the nucleic acids to target tissues, circulatory networks and to target cells (including inside target cells) prior to cleavage of the cargo molecule from the nucleic acid molecule.

In relation to polymers, the present invention provides a composition where nucleic acids comprise the structure of a matrix, where the matrix can be used as a detection device or as a drug delivery device. The nucleic acids of the matrix compositions comprise at least one aptamer region that specifically binds a ligand, and at least one nucleic acid cleaving region. The nucleic acids are linked to a matrix subunit, such as a polymer like polyacrylamide, whereby the nucleic acid linkages crosslinks the matrix subunits into a three-dimensional matrix. In this process of forming the matrix, one or more cargo molecules are included such that they are trapped in the interstitial spaces of the matrix, i.e., spaces between the matrix subunits and/or the nucleic acids. Upon binding of a ligand to an aptamer region, a conformational change in the nucleic acid causes the nucleic acid cleaving region to become active. As in the above-mentioned embodiments, the active cleaving region can cleave nucleic acids in cis and in trans, at specific nucleotide recognition sequences. The cis and/or trans cleavage therefore amplifies a response to the ligand. Further, since the nucleic acid-matrix subunit crosslinks maintain the assembly or integrity of the matrix, cleavage of the nucleic acids thereby results in the disassembly of the matrix. During disassembly, the interstitial spaces (inside the matrix) become exposed such that cargo molecules are no longer trapped within the matrix. Thus, this release of cargo molecules is a second order amplification of a response to a ligand. In a variation of this scheme, the polyacrylamide strands would be held together by a double stranded DNA made up of two short hybridizing single-stranded DNA molecules, one of which is an aptamer. The ligand would compete with the complementary DNA strand for binding to the aptamer sequence. When enough double stranded crosslinks had been disrupted, the gel would disassemble (Lin D. C. et al., J. Biomechanical Engineering, 2004, 126:104-110). No cleavage would be necessary in this case. The cargo molecules can be reporter molecules or protein enzymes when the matrix is envisioned in applications of detection, and the cargo molecules can be drugs when the matrix is envisioned in applications of drug delivery.

In the application of detection, the matrix can be present in a well, bead, slide, chamber or surface such that chromogenic, fluorogenic or luminogenic substrate molecules are bound or attached to the well, bead, slide, chamber or surface. The purpose of binding or attaching chromogenic, fluorogenic or luminogenic substrate molecules is to keep the matrix separated from the substrates such that these substrates will not migrate into the matrix and react with the reporter molecules. By maintaining separation of the matrix and the substrate molecules, the detection of fluorescent or chromogenic signals indicates the specific detection of a ligand. Alternatively, a well, bead, slide, chamber or surface can be designed such that the chromogenic, fluorogenic or luminogenic substrate molecules are separated from the matrix by a physical device, such as a membrane. In this embodiment, the membrane can have pores of a specified dimension. For example, the membranes are chosen that have pore sizes that are too small for chromogenic, fluorogenic or luminogenic substrate molecules (e.g., in polymerized form) to pass through but are large enough for reporter molecules to pass through. In this manner, once ligands cause the allosteric-dependent disassembly of the matrix, the reporter molecules are then free to pass through the membrane and catalyze reactions with the substrate molecules. In these application of matrix-mediated detection, the matrix and the substrates can be present in a well, slide, chamber or surface that is filled with solution.

If contemplated ligands are too large to pass through into the interstitial spaces of the matrix, then the invention provides a composition comprising at least two nucleic acids. The first nucleic acid comprises a first aptamer region that specifically binds a ligand and a first nucleic acid cleaving region, wherein binding of the ligand to the first aptamer region causes a conformational change in the nucleic acid such that the first nucleic acid cleaving region becomes activated. The second nucleic acid crosslinks matrix subunits thereby forming a matrix, and the second nucleic acid also comprises an aptamer region (“second aptamer region”) and a nucleic acid cleaving region (“second nucleic acid cleaving region”), where these regions have an allosteric relationship. In order to connect the response of the matrix to a ligand that is too large to enter the matrix, the first nucleic acid's aptamer region is designed to specifically bind a ligand of interest. When the ligand of interest binds to the first nucleic acid aptamer region, this causes the first nucleic acid region to become active, such that it cleaves the first nucleic acid molecules in cis and trans. This cleavage results in the creation of nucleic acid fragments that are small enough to pass into the interstitial spaces of the matrix. The second nucleic acid's aptamer region is designed to specifically bind the small nucleic acid fragments generated from the cleavage of the first nucleic acid. When a fragment specifically binds the second nucleic acid aptamer region, this binding causes a conformational change in the second nucleic acid such that the second nucleic acid's cleaving region is activated. The second nucleic acid's cleaving region can fragment or cleave the second nucleic acid in cis and trans, thereby causing the disassembly of the matrix and subsequent release of cargo molecules that have been trapped within the interstitial spaces of the matrix.

The present invention also provides methods and compositions of drug delivery that do not necessarily involve a nucleic acid cleaving region. Such a composition comprises a nucleic acid that has at least two aptamer regions. At least one aptamer region binds a drug and at least one aptamer region specifically binds a ligand. Upon specific binding of a ligand to an aptamer region, a conformational change in the nucleic acid causes the other aptamer region to release the drug. The nucleic acid can have two or more aptamer regions that bind the drug simultaneously in order to provide a tight binding affinity such that a premature release of the drug is prevented.

The compositions of the present invention provide methods of detecting ligands, including environmental and in vivo ligands. Such methods for detecting the presence of a ligand can comprise the steps of (i) contacting a sample with a composition of the present invention, and (ii) detecting whether or not cargo molecules are released from the compositions (i.e., cleaved off nucleic acids or released from matrices), wherein detection of the cargo molecules indicates the presence of the ligand in the sample. Other methods for detecting the presence of a ligand comprise: (i) contacting a sample with a composition of the present invention (or exposing the composition to an environment), and (ii) detecting whether there is an increase in fluorescence, wherein detection of the increase in fluorescence indicates presence of the ligand in the sample (or in the environment). Further methods for detecting the presence of a ligand comprise: (i) contacting a sample with a composition of the present invention (or exposing the composition to an environment), and (ii) detecting whether there is an increase in fluorescence, color, or chemiluminescence from the catalysis of a chromogenic, fluorogenic or luminogenic molecule, wherein detection indicates the presence of the ligand in the sample (or in the environment).

Ligands contemplated in the invention can comprise essentially any molecule or macromolecule, as aptamers can be designed and selected to bind almost any entity, including single small molecules, macromolecules, small-molecule drugs, nucleic acids, proteins, peptides, and microorganisms. Some specific entities contemplated by the invention include, but are not limited to, glucose, calcium, uric acid, cholesterol, vitamin D, creatinine, bilirubin, triglycerides, hormones, chemical toxins, bioterrorism agents, pollutants, irritants, allergens, immunogens, antigens, tumor-specific markers or antigens, cell or tissue-specific markers or proteins, and any combination thereof.

Nucleic Acid Functionalities

Single-stranded and double-stranded nucleic acids can adopt complex three-dimensional conformations that can exhibit specific binding abilities and even enzymatic activities. While proteins also exhibit these characteristics, the ability of nucleic acids to be chemically synthesized inexpensively and enzymatically amplified makes them molecules of choice as sensing and responding elements. The nucleic acids of the present invention can be either DNA or RNA, double-stranded or single-stranded.

Aptamers

Nucleic acid aptamers are single-stranded or double-stranded oligonucleotides that bind to a particular ligand with great affinity and selectivity. In the present invention, nucleic acid aptamer regions can range, for example, from about 15 to about 500 nucleotides, from about 40 to about 200 nucleotides, or from about 15 to about 100 nucleotides. The aptamers of the present invention can specifically bind almost any molecular or macromolecular entity as a ligand, such as ions, small organic molecules, nucleic acids, proteins, viruses, fungi and bacteria cells. Aptamers are created and selected using a combination of synthetic chemistry, enzymology and affinity chromatography.

By aligning short regions of complementary bases, DNA chains can form local double helical structures (secondary structures or stems) interposed with single-stranded loops. Additional interactions among the functional groups on the bases and with the sugar phosphate backbone of these loops produce a tertiary structure. The tertiary structure of each aptamer represents a unique 3-dimensional configuration, ultimately determined by its primary sequence.

The chemical synthesis of an oligonucleotide that incorporates a stretch of 25 nucleotides that are randomly selected from the 4 possible DNA bases will result in a population of 10¹⁵ different molecules of unique sequence and diverse structures. Because there are so many different chemical identities in such a population, it turns out that one can find a sub-population of these DNA molecules (10 to 1000, say) that will exhibit an affinity to almost any chemical structure one can formulate. These ligand-binding nucleic acid molecules are aptamers.

The ligands for apatmers can range from metal ions (for example, copper ions) to small organic molecules (e.g., Smirnov, I and Shafter, R. H., J. Mol. Biol., 2000, 296(1):1-5; Huizenga, D. E. and Szostak, J. W., Biochemistry, 1995, 34(2):656-65), to proteins (Feigon, J. et al., Chem Biol., 1996, 3(8):611-617; Griffin, L. C. et al., Blood, 1993, 81(12):3271-3276), to viruses (Tuerk, C. and MacDougal-Waugh, S., Gene, 1993, 137(1):33-39) and to bacteria (Kim, S. J. et al., Biochem. Biophys. Res. Commun., 2002, 291(4):925-931. Aptamers may be built of either DNA or RNA, and they are created and selected using a combination of synthetic chemistry, affinity chromatography and enzymology (Griffin, L. C. et al., Blood, 1993, 81(12):3271-3276).

The isolation of an aptamer that specifically binds to a target ligand consists of 3 steps: synthesis, selection, and amplification:

Synthesis: The chemical synthesis of aptamers can be conveniently and economically carried out by a commercial provider using an automated apparatus that is programmed to provide any specific sequence up to about 100 nucleotides or more. These commercial houses (e.g., Operon/Qiagen, Integrated DNA Technologies) can also modify the oligonucleotide sequences so as to put reactive functionalities like sulfhydryl, biotin, or primary amino groups at the 5′ or 3′ ends, which can be exploited to join these molecules to solid state scaffolds or to soluble polymers. If a substantial portion of the sequence is randomized, then a typical economical synthesis (0.05 to 0.5 mg) results in over 10¹⁵ different aptamers. A tiny fraction of this population will by chance have the ability to bind to the target ligand. Even if this fraction is minuscule (e.g., 1 in a million million) there will still be hundreds of representatives present in the population of 10¹⁵ molecules.

Selection: The small fraction of active molecules is then usually selected by affinity chromatography. The ligand is covalently attached to a solid support (e.g., agarose, polyacrylamide, cellulose) and then mixed with the starting aptamer population. After thorough washing, the bound aptamers are recovered under conditions that denature the DNA (e.g., heat, urea) or by competing for aptamer binding using the free ligand. The recovered active DNA will be present in a diminutive amount (e.g., 100 molecules, or ˜5 attograms). Moreover, it will be far from pure after this step: if the purification removes 99.9% of the non-specifically bound molecules, effecting a 1000-fold purification, 100 active molecules will still represent only 10⁻¹⁰ of the population. Further purification can be realized by iteration of this process, but an amplification step is required to generate a sufficient quantity of material to proceed.

Amplification: Amplification of nucleic acids can be accomplished by the polymerase chain reaction (PCR). For this purpose the original DNA population will have been synthesized with defined stretches of 20 nucleotides at each end, flanking the random region; these stretches act as priming sites for PCR. From sub-picogram amounts of DNA, PCR can generate microgram amounts by replicating all the template molecules presented to it. After several rounds (5-15) of this 2-part procedure (selection by affinity chromatography followed PCR amplification) the DNA population will consist mostly of tight binding species, as evidenced by most of the material binding to the affinity chromatography material. At this point a small number of individual molecules are purified by cloning in a plasmid in E. coli, and their exact sequence is determined. Binding constants of the pure aptamers to the ligand can be measured and their specificity tested by examining binding of related molecules.

Refinement: Improvement in the specificity and affinity of the best aptamer can now be accomplished using further genetic variation. A new set of aptamers is synthesized using as a framework the sequence of the best selected aptamer. This second synthesizes a mutagenic procedure: at each position only 70% of the new molecules receive the original nucleotides, the remaining 30% is split evenly among the other 3 nucleotides. In this way a new set of 10¹⁵ molecules is produced, each a variation on the theme represented by the starting selected aptamer. Selection and amplification proceeds as above. In these rounds, however, further selective pressure can be applied. For example, if greater affinity is desired, then aptamers that are easily and quickly eluted from the affinity chromatography material can be eliminated, selecting only those molecules that require extensive incubation with free ligand to elute (i.e., once bound, they have a long on-time). Analogously, specificity can be selected by washing with a related ligand and discarding aptamers that are eluted.

Additionally, the SELEX (Systematic Evolution of Ligands by Exponential Enrichment) method allows for the identification of nucleic acids that can specifically bind ligands. A candidate mixture of single stranded nucleic acids having regions of randomized sequence is contacted with a target ligand compound and those nucleic acids having an increased affinity to the target are partitioned from the remainder of the candidate mixture. The partitioned nucleic acids are amplified to yield a enriched mixture with which the process is repeated. For example, see U.S. Pat. Nos. 5,270,163; 5,475,096; 5,567,588; 5,595,877; 5,637,459; 5,670,637; 5,683,867; 5,688,935; 5,696,249; 5,705,337; 5,723,289; 5,723,592; 6,261,774; 6,465,189; 6,482,594; and 6,569,620.

Ribozymes and DNAzymes

Aptamers are a broad class of molecules that encompass nucleic acids having just specific ligand binding ability and nucleic acids having enzymatic activity, where such enzymatic activity implicitly involves a binding ability. For the present invention, the invention uses the term “aptamer” to mean a nucleic acid region that can specifically bind a ligand. However, this does not mean that an aptamer region in the present invention cannot have functions in addition to specific ligand binding.

Oligonucleotides not only have the ability to bind specific ligands, but can catalyze a chemical reaction involving the ligand. RNA-based enzymes (ribozymes) exist in nature, and for the most part they exhibit RNA-cleaving activity (Zhen, B. et al., Sheng Wu Hua Xue Yu Sheng Wu Wu Li Xue Bao (Shanghai), 2002, 34(5):635-642). DNA-based enzymes (DNAzymes) that cleave RNA or DNA at specific sequences have also been isolated through selection and amplification. For instance, a DNAzyme that exhibits DNase activity in the presence of copper ions has been isolated (Carmi, N. and Breaker, R. R., Bioorg. Med. Chem., 2001, 9(10):2589-2600). DNAzyme activities in addition to RNA and DNA cleavage include DNA ligation (Soukup, G. A. and Breaker, R. R., Trends Biotechnol., 1999, 17(12):469-476), DNA capping (Hamaguchi, N. et al., Anal. Biochem., 2001, 294(2):126-131), phosphorylation (Soukup, G. A. and Breaker, R. R., Trends Biotechnol., 1999, 17(12):469-476), acyl coenzyme A-transferase activity (Doudna, J. A. and Cech, T. R., Nature, 2002, 418(6894):222-228) and peroxidase activity (Li, Y. and Breaker, R. R., Curr. Opin. Struct. Biol., 1999, 9(3):315-323). Thus, DNAzymes and ribozymes can catalyze several different reactions and they can act as RNA and DNA endonucleases (DNases), kinases, ligases, capping enzymes, promoters of amino acid activation, acyl transfer and the Diels-Alder reaction. Also of relevance to this invention are DNAzymes that catalyze the cleavage of DNA chains at defined sequences. As in the case of allosteric aptamers, the catalytic activity of ribozymes and DNAzymes can be made dependent on the ligand binding state of an independent aptamer residing in the same molecule.

When an aptamer domain binds a ligand it can effect a conformational change in the whole molecule that either activates or inactivates the second aptamer (Soukup et al., J. Mol. Biol., 2000, 298(4): 623-632; Soukup and Breaker, Trends Biotechnol., 1999, 17(12): 469-476); this relationship between the conformational change induced by ligand binding to a first aptamer domain resulting in a change of function for the second aptamer domain (and potentially additional aptamer domains) or for another nucleic acid functional domain (for example, a nucleic acid cleaving region) is an allosteric relationship. For example, the binding of a ligand to an aptamer domain can induce an increase in the fluorescence of an adduct bound to the oligonucleotide due to a reduction in quenching (Hamaguchi, N. et al., Anal. Biochem., 2001, 294(2): 126-131). Moreover, this allostery or allosteric control can be selected for in much the same way as simple ligand binding, since the individual aptamer domains can act as semi-autonomous modules (Cairns, M. J. et al., Nat. Biotechnol., 1999, 17(5):480-486). However, prior reports of allosteric control do not solve the problem of providing an amplified response to low levels of ligand, where a response is often too weak for practical applications due to a linear relationship between the ligand levels and the response. Further, these prior reports do not mention the use of nucleic acid functionalities to provide a controlled drug delivery method.

Matrices

The nucleic acid molecules of the present invention can be used as components in a larger composition, whereby the aggregation of component nucleic acids into a larger whole can provide multivalency and a mechanism to amplify their activity. For example, multiple nucleic acid molecules can be bound to a bead, where the nucleic acid(s)-bead composition itself can be crosslinked or attached to other nucleic acid(s)-bead compositions such that a matrix is formed. Alternatively, nucleic acids can be structural components of a gel-based matrix, such that the nucleic acid cross-link gel monomers and polymer subunits together. This gel-based matrix would also provide a route for signal transduction and act as a sink/storage area for the response. The matrix compositions of the present invention include the use of polymers and polymer gels. In one embodiment, the present invention provides “smart nanogels” by incorporating custom-tailored nucleic acids, preferably DNA, with aptamer and/or enzymatic functional regions (that have been created by evolution in the test tube, identified and isolated for ligand binding) as an integral part of a polyacrylamide matrix. The DNA aptamers will bind their ligand with great affinity and specificity and the gel matrix will respond in a well characterized way to external perturbations. The range of possible ligands is broad; success has been demonstrated for metal ions, drug molecules, proteins, and even whole bacterial cells. A new class of materials based on “sensor-actuator” dynamics takes advantage of allosteric effects induced by combining different nucleic acid functional regions with specific applications as nanoscale controlled release deliverers.

“Nanogels” are nano-sized cross-linked but soluble polymeric particles or bulk composite gels (with possibly inorganic and biological entities distributed in it) having induced nanometer sized heterogeneities (e.g., distribution of nanoparticles). In the present invention, biocompatibility and biodegradability are criteria for selection of the polymer type. Chemical reactivity can be induced in these nanogels by choosing appropriate matrix subunits, such as monomers, thus making it possible for the nanogel to include or exclude other materials and to make them sensitive to many different kinds of stimuli. This combination of physical and chemical properties makes these nanogels an ideal choice for use as a vehicle in the development of smart systems and products based on sensor-actuator dynamics. The nanogels can be sensitive to perturbations such as electrical and thermal change, in addition to the chemical sensing described within.

The matrices of the present invention can comprise one or more different types of matrix subunits. Matrix subunits of the present invention, include, but are not limited to, agarose, polyacrylamide, polysaccharide, polystyrene, polypropylene, polyethylene, polyurethane, polysiloxane, polymethyl methacrylate, polyvinyl alcohol, polyethylene, polyvinyl pyrrolidone, or any combination thereof.

Integrating nucleic acids into nanogels provides the opportunity to develop novel “smart” biomaterials which may be designed to be a reporting biosensor or a means of sequestering and removing toxins as well as a biosensing drug delivery vehicle.

Cargo Molecules

As used herein, “cargo molecules” means molecules that are attached or contained within the compositions of the present invention. For example, reporter molecules, such as enzymes that catalyze reactions with fluorogenic, luminogenic, chromogenic or electrical-signal generating substrate molecules, that are attached to nucleic acids are considered cargo molecules. Also, reporter molecules or drug molecules that are contained within the interstitial spaces of the matrix are considered cargo molecules.

Reporter Molecules and Their Substrates

The present invention encompasses nucleic acids that are have attached or bound reporter molecules or protein enzymes. Further, the present invention encompasses matrices containing reporter molecules or protein enzymes. Reporter molecules include, but are not limited to, enzymes that can catalyze reactions with fluorogenic, luminogenic or chromogenic substrate molecules, for example, horseradish peroxidase, alkaline phosphatase, acid phosphatase, β-galactosidase, luciferase and β-glucuronidase. The chromogenic, fluorogenic and luminogenic substrate molecules include, but are not limited to, derivatives of: 5-bromo-4-chloro-3-indolyl phosphate; 2,2′-azino-di[3-ethyl-benz-thiazoline sulfonic acid; 3,3′,5,5′-tetramethylbenzidine; o-phenylenediamine; p-nitrophenyl-phosphate; o-nitrophenyl-β-D-galactopyranoside; chloro-phenolic red-β-D-galactoopyranoside; or NADP glucose 6-phosphate, luciferin and ATP, fluorescein diphosphate; dimethylacridinone phosphate; ρ-hydroxyphenylacetic acid; 3-(ρ-hydroxyphenyl)propionic acid; 4-methylumbelliferyl phosphate; 6,8-difluoro-4-methylumbelliferyl phosphate; 4-methylumbelliferyl-β-D-galactopyranoside; fluorescein di-β-D-galactosidase; 4-methylumbelli-feryl-galactoside 6-sulfate; 1,2-dioxetanes; luminol; coeleterazines; luciferins; acridines; and metal ions.

Other Cargo Molecule Enzymes

Cargo molecules can also be enzymes that can cleave or release charged molecules that are bound to the exterior of CNT transistors. In this manner, enzymes can alter the conductance properties of CNT transistors. Cargo molecules can also be enzymes that can cleave derivatized fluorogenic peptides at a region that does not affect the fluorogenic capability of the peptide. For example, derivatized fluorogenic peptides can be designed to comprise a fluorogenic molecule, a region that is specifically cleaved by an enzyme and a region that can tether this whole derivatized fluorogenic peptide to a solid surface. An enzyme will be therefore able to cleave the derivatized fluoregenic peptide and release the fluorogenic molecule region without negatively affecting fluorogenic ability. For purposes of the present invention, the term “fluorogenic molecule” includes derivatized fluorogenic molecules/peptides.

Examples of enzymes contemplated by the invention include, but are not limited to trypsin, subtilisin, hyaluronidase, chitinase, cellulose, phospholipase C, or DNA restriction enzymes. Substrate molecules for these enzymes include, but are not limited to, anthraniloyl-Lys-p-nitroanilide, peptide with a subtilisin cleavage site, hyaluronic acid, chitosan, carboxymethylcellulose, dipalmitoyl-phosphatidyl-inositol-diphosphate, or double stranded DNA.

Drug Molecules

The present invention encompasses drug molecules that can be attached to nucleic acids or delivered in matrices. Drug molecules include, but are not limited to, insulin, cytokines, antibodies, hormones, small-molecules, antibiotics, anti-histamines, steroids, enzyme agonists, receptor agonists, enzyme antagonists, receptor antagonists, and any combination thereof.

Sensitivity

As stated, the present invention is capable of detecting very small amounts of ligand. For example, aptamers can bind to ligands with dissociation constants of 1 nM. In relation to this dissociation constant, and in relation to a well having a volume of 10 microliters, then the nucleic acid molecules of the present invention can have a sensitivity of detection of about 10 femtomoles of ligand, which is still 6×10⁹ molecules. Further, since wells can be miniaturized, the degree of sensitivity can be increased in relation to the degree of miniaturization of the well. For example, for a well with a 1 nanoliter volumes (100 um×100 um×100 um), this would afford a 10,000-fold increase in sensitivity, to a detection sensitivity of about 1 attomole (10⁻¹⁸ moles; 600,000 molecules). Assuming that each ligand molecule results in the release of at least one reporter molecule, 600,000 reporter molecules can thereby be released. However, one can detect, by fluorescence assay, one molecule of alkaline phosphatase (a single alkaline phosphatase molecule can be detected, Craig, D. B. et al., J. Am. Chem. Soc. (1996) 118: 5245-5253). Thus, the limit of detection of the present invention can therefore be as small as 1 ligand molecule.

Most aptamer-based ligand detection methods are based on producing a one-to-one signal by an aptamer: when one ligand becomes bound, one fluorescent molecule is activated (e.g., Seetharaman, S., et al., Nat. Biotechnol., 2001 19:336-341). Even when the catalytic activity of a ribozyme or DNAzyme is exploited by allowing one activated ribozyme to produce more than one fluorescent molecule (e.g., Frauendorf, C. and Jaschke, A., Bioorg. Med. Chem., 2001, 9:2521-2524; Brown, A. K. et al., Biochemistry, 2003, 42:7152-7161), the activity is limited by the low turnover number (k_{cat}, molecules of substrate converted per molecule of enzyme per unit time) of nucleic acid-based enzymes, typically less than 1 per minute (Joyce, G. F., Annu. Rev. Biochem., 2004, 73:791-836). In contrast, protein enzymes often have turnover numbers thousands of times faster than this, including subtilisin (Zhao, H and Arnold, F. H., Protein Eng., 1999, 12:47-53; Stambolieva, N. A. et al., Arch. Biochem. Biophys., 1992, 294:703-706), an industrial enzyme. Thus the strategies proposed in the present invention include the potential of increasing sensitivity of detection by over 3 orders of magnitude when using cargo molecules that are enzymes which cleave fluorogenic molecules (where the cleavage does not affect the fluorescent ability of the molecules; i.e., fluorogenic molecules can be peptides that have been derivatized to have an immobilization group that tethers the fluorogenic molecule and a fluorogenic dye group) that are tethered to blocking surfaces.

Thus, several embodiments in the present invention provide for additional methods to improve detection sensitivity. First, a fluorescent background inherent in the fluorogenic substrate molecules can be eliminated by immobilizing these molecules on an area of gold surface (or other blocking surface). The exciting light will not penetrate the gold and so no signal will be contributed by the fluorogenic substrate molecule itself. After it is cleaved, the highly fluorescent products will diffuse away from the gold surface and so be exposed to the exciting radiation. Background signals can be reduced by requiring the cleavage or relaxation of multiple tethers to release the protein enzyme. This type of background signal can also be reduced by incorporating 2 inhibitory aptamers, requiring each to be bound by ligand to effect the activation of the catalytic center (Jose, A. M. et al., Nucleic Acid Res., 2001, 29:1631-1637). Finally, additional rounds of selection focused on improving sensitivity can be applied; such experiments have improved the ratio of activated vs. background activity of a ribozyme more than an order of magnitude to over 3000 (Soukup, G. A. et al., J. Mol. Biol., 2000, 298:623-632).

The robust sensitivity of the methods contained in the present invention will aid in the development of small-scale sensing devices such as a wireless handheld unit. Such a device could be used to detect bioterrorism agents including small molecules such as nerve gases and explosives as well as noxious proteins such as ricin and botulism toxin. It could also be applied to monitoring a variety of environmental pollutants and even be adapted for the analysis of body fluids.

It is to be understood and expected that variations in the principles of the invention herein disclosed in an exemplary embodiment can be made by one skilled in the art and it is intended that such modifications, changes, and substitutions are included within the scope of the present invention.

The examples set forth below illustrate several embodiments of the invention. These examples are for illustrative purposes only, and are not meant to be limiting.

### EXAMPLE 1

### Detection of Ligand-Aptamer Interactions Using a Carbon Nanotube

The commercial protease subtilisin can be used as a cargo molecule attached to a nucleic acid. Subtilisin has great stability, broad substrate specificity, and high activity (kcat of ˜>100 per second on many substrates (Zhao, H. & Arnold, F. H., Protein. Eng., 1999, 12:47-53; Stambolieva, N. A. et al., 1992, Arch. Biochem. Biophys., 294:703-706)). The peptide N-acetyl-glu-glu-ala-glu-glu-ala-glu-glu-ala-ala-pro-phe-AHA₆-pyrene (SEQ ID NO: 1) is a substrate cleaved by subtilisin and can be used to coat the carbon nanotube. The peptide should adhere strongly to the carbon nanotube via the pyrene group on its carboxyl end (Petrov, P. et al., 2003, Chem. Commun. (Camb.), 2904-2905). The six glutamate residues should impart a strong negative charge to the carbon nanotube. Upon ligand binding to the aptamer, the cargo molecule subtilisin will cleave the peptide after the phe residue. The freed peptide will leave the pyrene on the carbon nanotube but the freed peptide itself should no longer bind, its amine group being masked by acetylation. As the charged peptide is released, the resulting changes in the conductance properties of the carbon nanotube can be detected as a change in the voltage/current relationship of the field effect transistor (FIG. 7).

In a variation on the above example, the protease cargo molecule would be tethered to a carbon nanotube by an aptamer that forms the stem in a stem-loop DNA structure; this molecule would be surrounded by hundreds of substrate molecules on a 10 um carbon nanotube. In the presence of ligand, the aptamer would be induced to de-hybridize from its complementary strand, binding the ligand as a mutually exclusive alternative (Nutiu, R. & Li, Y., 2004, Chemistry, 10:1868-1876). The DNA, now in a linear conformation, would extend, cleaving substrates over a long distance on the carbon nanotube (FIG. 11)

Modifier molecules can be covalently attached to carbon nanotubes (Nutiu and Li, 2004; Zhao and Arnold, 1999); strong attachment can also be effected by hydrophobic forces (Stambolieva et al., 1992; Petrov, P. et al., 2003) or a combination (Laska, M. et al., 2003, Chem. Senses, 25:47-53). Both aptamer/DNAzymes and substrates can be attached to the same nanotube. Other negatively charged polymers (polysaccharides and lipids) that are vulnerable to enzymatic digestion are alternatives to peptides; several are listed in Table 1 below. The last line in the table suggests a different strategy: direct electrical detection of charge transfer changes in a DNA aptamer brought about by the conformational change induced by ligand binding (Zhao and Arnold, 1999; Belgrader, P. et al., 2003, Anal. Chem., 75:3446-3450). It may be that adding sticky charged molecules to a carbon nanotube will constitute a more sensitive method than removing such groups. In this case, the protease could be used to free a pyrene-derivatized highly charged peptide from a solid state support adjacent to the carbon nanotube, allowing it to diffuse to the carbon nanotube, adhere and exert its effect.

TABLE 1

| CNT modifer | CNT attachment | Modifying agent |
|---|---|---|
| 18-mer oligopeptide with a | N-terminal amino or | subtilisin |
| proximal subtilisin cleavage | N-terminal pyrene | |
| site followed by 6 | | |
| glutamic acid residues | | |
| Hyaluronic acid | Terminal pyrene | hyaluronidase |
| Chitosan | Terminal pyrene | chitinase |
| Carboxymethylcellulose | Sparse internal pyrenes | cellulase |
| Dipalmitoyl-phosphatidyl- | Hydrophobic forces | Phospholipase C |
| inositol-diphosphate | | |
| Double stranded DNA | Terminal pyrene | Restriction enzyme |
| Aptamer DNA | Terminal pyrene | Ligand directly |

### EXAMPLE 2

### Isolation of an Allosteric DNA Molecule that Responds to Glucose

A population of random DNA sequences can be provided and a subset selected that is able to bind glucose. However, in this case, a DNA aptamer that binds the glucose disaccharide cellobiose (glucose-beta-glucose) has already been described (Yang, Q., et al., Proc. Natl. Acad. Sci. USA, 1998, 95(10): 5462-5467). This aptamer was isolated on the basis of its ability to bind to a column of cellulose, which is polymerized cellobiose; cellulose can also be viewed as polymerized glucose. This aptamer binds cellobiose tightly but not other glucose derivatives. The cellobiose aptamer can be used as starting material for the generation of a large number of genetic variants of this sequence by having a population of molecules synthesized with only 70% fidelity (doped synthesis); i.e., there is a 30% chance of having any one of the 3 alternative DNA bases at each position. The entire molecule is 89 nucleotides (nt) in length, with primer binding sequences of 20-25 nt flanking the 44 nt aptamer sequence. Fifty μg of this DNA consists of 10¹⁵ molecules, almost all of which are different variations on the cellobiose binding theme.

Aptamers that exhibit glucose binding ability are selected from this population using affinity chromatography on solid fibrous cellulose (and/or amylose-agarose beads from Sigma; amylose is polymerized glucose-alpha-glucose). After allowing the DNA and cellulose to interact, the unbound molecules are washed away and the bound DNA molecules are then eluted with glucose (rather than cellobiose). Concentrations of glucose that mimic blood levels after a meal (as high as 20 mM) are used in the presence of 2 mM Mg⁺⁺. The eluted aptamers are then amplified using the polymerase chain reaction (PCR) to regenerate enough material with which to continue. The PCR product is then exposed to cellulose to allow the amplified aptamer-enriched DNA to bind once again. After 5 to 15 reiterations, most of the DNA is binding to the column and is eluted with glucose. At this point the desired molecules are abundant enough (>1 in 10) to clone and test as pure species.

The final DNA population is inserted into a plasmid and used to transform bacterial cells (E. coli). Individual bacteria that have received a single DNA molecule are isolated and grown in large numbers. The aptamer from each bacterial clone is then easily isolated in large quantities using routine, known methods.

If necessary, further refinement of aptamer properties may be carried out by random mutagenesis of an effective aptamer. Here the DNA sequence is synthetically mutated again, to introduce a substantial level of random base substitutions within the framework of the original sequence, this time only about 10% total at each position. Once again a large number of different sequences are generated, and aptamers with the desired properties isolated by affinity chromatography and amplified by PCR. Such refinement may be necessary if the aptamer does not exhibit sufficient selectivity for glucose, for example. This will be evident if it can be eluted from cellulose with other sugars, such as fructose or ribose. In this case, the selection for selectivity is straightforward: the cellulose with the bound DNA will be first exposed to the non-glucose sugars and DNA molecules that are eluted will be discarded. The glucose-specific aptamers are then eluted using glucose.

### EXAMPLE 3

### Isolation of an Allosteric Nucleic Acid that Cleaves in the Presence of Glucose

A new stretch of 40 random nucleotides is synthesized adjacent and upstream of the glucose aptamer described in Example 2 (i.e., on its 5′ side). From this population, molecules are selected with DNase activity, i.e., molecules that are able to cleave single stranded DNA in cis or trans. Such cleaving DNAzymes have been isolated in two different laboratories in the past (Carmi, N. and R. R. Breaker, Bioorg. Med. Chem., 2001, 9(10): 2589-2600; Sheppard, T. L. et al., Proc. Natl. Acad. Sci. USA, 2000, 97(14): 7802-7807). Since such DNAzymes can be self-destructive, (i.e., self-cleavage or cis cleavage), their activity must be modulatable to allow their isolation, i.e., they must be first isolated in their inactive state. Such modulation is in fact what is desired for selection: the DNAzyme should be inhibited by the (empty) glucose aptamer domain in the absence of glucose. On the other hand, it should be activated when glucose binds to its aptamer domain. Such allosteric modulation involves a change in the shape and activity of the aptamer that results from binding with a regulatory substance at a site other than the catalytic one. This allosteric behavior of the DNAzyme is illustrated in FIG. 6.

About 10¹⁵ new bipartite (nucleic acid molecules having both glucose aptamer and a 40 base-pair region that would include DNAzyme functional regions) molecules are synthesized with a biotin molecule added to the 5′ end. These molecules will then be mixed with agarose beads bearing the protein streptavidin. Streptavidin binds biotin with great affinity, so the DNA will become immobilized at this step. The immobilized DNA will then be exposed to 20 mM glucose (plus Mg⁺⁺). The glucose will bind to the glucose aptamer domain. Some of these molecules have self-cleaving ability. After cleavage, these molecules are released from the beads and are isolated. These released molecules are truncated at their 5′ ends. Some of them have been cleaved close to the 5′ end. Indeed, a short sequence is incorporated that is known to be a substrate for a DNAzyme in case that represents a favorable general substrate for cleavage. These eluted molecules are then amplified by PCR, using as a 5′ primer a DNA oligomer that incorporates the substrate sequence within its priming sequence. Thus the cleaved substrate sequence is regenerated for the next round of selection. The primer also has a biotin residue at the 5′ end. Following PCR the DNA is once again attached to streptavidin beads. The whole process is reiterated 5-15 times, until most of the DNA is cleaved in the presence of glucose. The selection scheme is illustrated in FIG. 7. Once again, refinement may be necessary. Here, in addition to overall doped-synthesis mutagenesis, one can vary the distance and the sequence between the catalytic and glucose-binding domains to isolate molecules that are more responsive to glucose or with higher catalytic activity than the original isolate.

Insulin is then attached to the nucleic acids at the terminus that becomes cleaved upon glucose-aptamer mediated activation of the cleaving region.

Alternatively, insulin is not attached, but rather, the nucleic acids are crosslinked to polymers such that a matrix is formed with insulin trapped within the interstitial spaces. This matrix is then used as a controlled-insulin delivery device. The allosteric aptamer-DNAzyme nucleic acids are incorporated into gels as cross-linking molecules that produce gelation of the polyacrylamide polymers. The binding of the ligand triggers a disassembly of the gel with the concomitant release of the insulin cargo that is preloaded in the gel during its synthesis. This DNA cross-link is cleaved only upon binding glucose to the aptamer domain. Loading of insulin is achieved through non-covalent interactions between the molecule and the polymer matrix. Additionally, the loaded molecules are immobilized in the polymer gels via formation of a biodegradable covalent bond between the drug moiety and the polymer matrix. This approach of using dispersed gels results in high loading capacity and provides an advantage from a regulatory perspective because polymer gels are synthesized and evaluated in the absence of the loaded molecule (Vinogradov, S. V. et al., Adv. Drug Deliv. Rev., 2002, 54(1): 135-147).

Since insulin is needed in such small amounts (<10 nM), a dosage of only a few microliters of beads (i.e., nanogel) should suffice. For example, if a bead 100 nm in diameter is 50% interstitial water and is loaded with only 10% efficiency from a 1 M solution (6 mg/ml), each bead will contain 30000 insulin molecules. Five liters of human blood at 10 nM requires 3×10¹⁶ insulin molecules, or 10¹² beads of volume 10⁻¹⁵ ml each for a total of 1 microliter of packed beads.

### EXAMPLE 4

### Isolation of a Nucleic Acid Having Multiple Aptamers

An attractive feature of the gel disassembly scheme is its potential for general applicability. Aptamer domains often act autonomously and can be viewed as portable modules. Thus with only a few adjustments it may be possible to substitute a different aptamer domain in the allosteric DNAzyme to create matrices that could deliver any protein in response to any ligand. An alternative scheme for controlled delivery of insulin involves nucleic acid molecules having multiple aptamer domains. In the alternative scheme, the matrix, if used, is used more passively as a carrier to hold multiple nucleic acids on particles whose size will control their clearance rate.

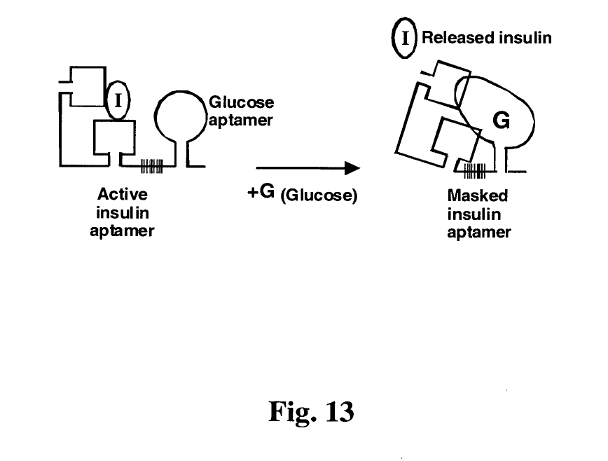

Three aptamers are combined on one DNA molecule, the aforementioned glucose aptamer of Example 2 and two new aptamer region sequences selected for binding to insulin. The latter two aptamer regions will be selected once again by affinity chromatography, passing a random library of DNA molecules over a column of immobilized insulin. Among the final candidates, two aptamers are chosen that exhibit the highest affinity for insulin and that do not compete with each other for binding; i.e., they bind to different epitopes on the surface of the insulin molecule. The two insulin aptamers are then combined with the glucose aptamer with a short region of a random spacer sequence between them. Molecules are then selected that retain the ability to bind insulin despite the presence of the (empty) glucose aptamer (without glucose). Because the two insulin aptamers are combined, the net result is extremely tight insulin binding, since individual aptamer-protein complexes typically have binding constants in the nanomolar range. Such tight binding is important to prevent the premature release of insulin over time in the absence of high glucose levels (background). The bound DNA molecules are then eluted by adding 20 mM glucose and Mg⁺⁺. Those molecules that are recovered (and are then amplified by PCR, as described above) are those that exhibit the desired allosteric property: a glucose-induced conformational change that compromises the binding of insulin to the insulin aptamers, as illustrated in the FIG. 13.

An advantage of this design is that it is reversible: once the glucose concentration falls, the nucleic acids are able to bind insulin and thus reduce or stop its hormonal action. Perhaps the most important aspect of this embodiment is that gels containing these insulin-glucose aptamer nucleic acids must release their insulin at high post-prandial glucose concentrations of 20 mM but not do so at fasting levels of blood glucose of 3 to 7 mM. Thus, a typical high-affinity aptamer would not be appropriate for this application. However, the ease of selection inherent in the affinity chromatography step of aptamer isolation lends itself well to the isolation of molecules that could exhibit this degree of discrimination. The evolution of a discriminatory aptamer may be facilitated by the inclusion of two glucose binding aptamers in the DNA, fostering the possibility of cooperative interaction and an S-type binding curve. Alternatively, we could select a glucose aptamer with high affinity that promotes insulin binding and then add a second glucose aptamer with low affinity that disrupts this process.

In this example, we have chosen to use DNA aptamers rather than RNA aptamers because DNA is chemically very much more stable than RNA, and there is no evidence that DNA is any less capable of forming aptamers than RNA. RNA is also less stable in the bloodstream, being subject to degradation by RNases. However, DNases (especially 3′ exonucleases) are also present in the blood, compromising the stability of DNA aptamers. These can be blocked by modification of the 3′ end of the aptamer (Brody, E. N. and L. Gold, J. Biotechnol., 2000, 74(1): 5-13). Free aptamers, being of relatively low molecular weight, are rapidly cleared from the blood by organ extraction (Dougan, H., et al., Nucl. Med. Biol., 2000, 27(3): 289-97). The location of the cross-linking nucleic acids in the interior of a nanogel affords protection from degradative enzymes, as unlike glucose, nucleases will be too large to permeate the gel. Moreover, because the 3′ end of the aptamer is in a covalent bond to polyacrylamide, it is resistant to 3′ exonucleases. If stability is still a problem, nucleic acids can be modified to make them resistant to enzymatic degradation. A common procedure is to substitute 2′ amino- or 2′ fluoro-nucleosides for deoxynucleosides during chemical synthesis (Brody, E. N. and L. Gold, Reviews in Molecular Biotechnology, 2000. 74(1): 5-13). These modified nucleotides can act as substrates for the Taq DNA polymerase used in the PCR step. The clearance issue has been more problematic, but success has been achieved by conjugating aptamers to polyethyleneglycol (PEG) of moderate molecular weight (e.g., 40,000 daltons) (Brody, E. N. and L. Gold, Reviews in Molecular Biotechnology, 2000. 74(1): 5-13; Watson, S. R. et al., Antisense Nucleic Acid Drug. Dev., 2000, 10(2): 63-75). Our use of soluble matrices or nanogels should provide similar clearance delay, and our ability to vary the size and to decorate the exterior of the nanogels allows flexibility to try various modifications to improve in vivo lifetime. Moreover, nanogels are not limited to using polyacrylamide as the subunit; alternative polymers may prove superior in terms of retention.

### EXAMPLE 5

### Synthesis of Matrices

The general methodology to be employed in this example is a familiar one using reverse microemulsion polymerization (although for the present invention, any standard method for emulsion can be used, as there are numerous surfactants that would be sufficient for emulsification). Monomers are dissolved in the water droplets of an inverse microemulsion (water/toluene) stabilized by the surfactant AOT and subsequently polymerized using γ-radiation (Wilk, R. J. “Synthesis and Characterization of Pyrene-Labeled High Molecular Weight Polyacrylamide Polymers and Microgels,” Doctoral Thesis Columbia University 1994). Such nanogels possess polyelectrolyte segments with numerous reactive groups readily available for subsequent conjugation with ligands. Using this method it has also been demonstrated (Liu, F. et al., “Polyacrylamide Microgels Synthesis, characterization and Modification for Overdosed Drug Detoxification,” in Particles 2002, Orlando, Fla., 2002; Liu, F. et al., “Synthesis and modification of polyacrylamide nanogels for drug delivery applications,” in ACS 26th National Conference, Chicago, Ill., 2001; and Liu, F. et al., “Modified polyacrylamide nanogels for overdose drug removal. in NSF engineering research center for particle science and technology,” IAB meeting, Gainesville, Fla., 2002) that through incorporation of a few percent reactive monomers, e.g., acryloxysuccinimide esters, in the polymerization process, nanogels can result that have their exterior decorated with active esters. These esters have been shown to react with a variety of nucleophiles including primary amines. Thus using the nucleophilic substitution route the nanogels can be modified to incorporate various functionalities.

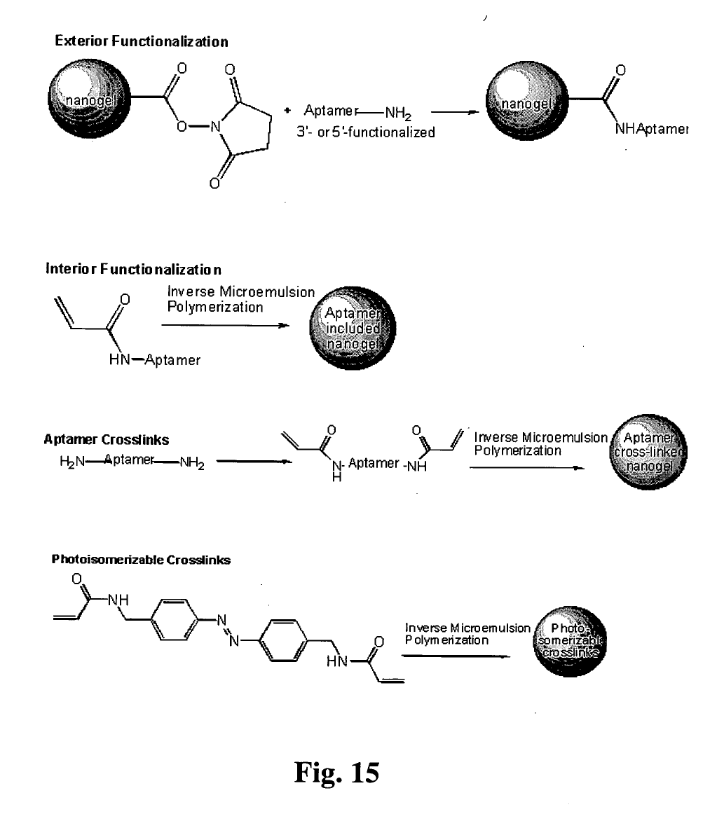

Through this postgrafting strategy primary amine functionalized aptamers are reacted with the exterior of the nanogels. These amine-terminated aptamers are commercially available from Operon/Qiagen in either 3′ or 5′-functionalized versions. These reactions, shown schematically in FIG. 15, positions aptamers on the exterior of the nanogel.

Linker regions can be added between the aptamer and/or enzymatic regions and the acrylate polymerization precursors. This can be achieved by using either elastic linkers such as oligomeric polyethylene oxide or polyisoprene [Zubarev, E. R. et al., Science, 1999, 283: 523-526]. In order to insure that the nucleic acids are incorporated in the interior of the “nanosphere” matrices, the ratio of the surfactants, solvents, nucleic acids, and linkers are systematically varied to find optimal conditions.

By using the methodology developed for the interior functionalization, we can incorporate acrylate monomers at both the 5′- and 3′-ends of the aptamers. This cross-linking reagent can then be incorporated into the inverse microemulsion polymerization. The aptamer bis-acrylates can be conveniently prepared from aptamers that are commercially available in their derivatized form with primary amines at both ends. Using standard coupling procedures these can be linked to the acrylate precursors. Again, the ratio of the surfactants, solvents, aptamers, and linkers are systematically varied to find optimal conditions

Incorporation of fluorescent tags and ESR (electron spin resonance) probes into the nanogels that have been derivatized with aptamers or otherwise, serve as analyzers of the matrix environment to allow the determination of structures. They can be conveniently synthesized by known methods and the ones outlined above from commercially available fluorescent and ESR tags that have amino-functionality that will allow them to react with activated esters on the interior and exterior of the nanogel (Ottaviani, M. F. et al., Helv. Chem. Acta, 2001, 84: 2476-2492). It is presumed that these reagents unlike the aptamers will be able to penetrate into the nanogel.

Additionally, nanogels can be synthesized that have cross-linkers that can change their size when photoisomerized. The cross-linker in FIG. 15 details the reaction. When in its trans form (shown) the cross-linker is extended and when in the cis form it is C-shaped. These molecules can be conveniently synthesized from commercially available precursors using the procedures outlined above.

By tuning the chemical constitution of the polymer backbone, different and useful properties can be installed into the nanogels.

In order for these nanogels to be used in the blood system, the particles can be about 5 nm to about 100 nm in diameter, or a size small enough to pass through the kidney membrane. Efforts to control particle size will be necessary; alternatively a biodegradable cross-linker can be incorporated.

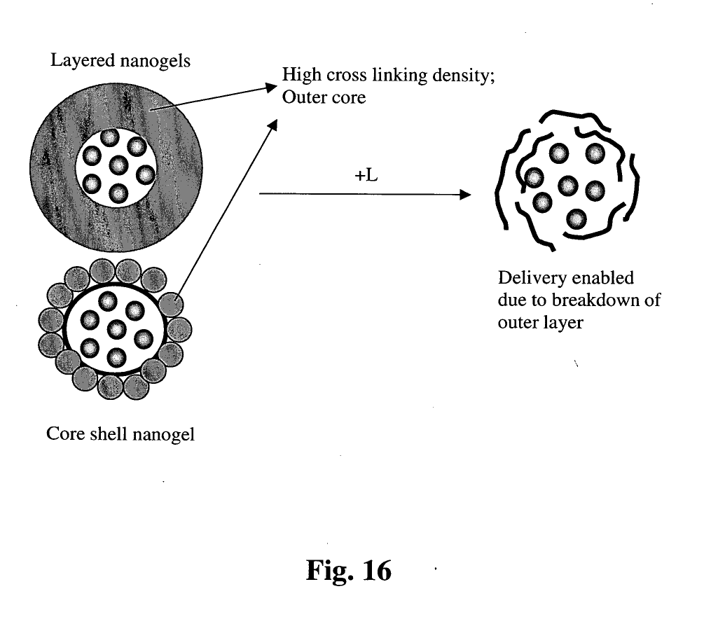

Layered nanogel structures are also built (FIG. 16). The interior core contains the deliverable molecules in a low cross-linked density high porosity nanogel. The exterior (cortex) is made up of high cross-linked density nanogels with nucleic acids comprising aptamers and DNAzymes acting as the cross-linkers. On detecting a binding ligand the action of the cleaving region leads to the breakdown of the outer cortex, which leads to the delivery of the molecules due to the high porosity of the interior. Previous studies report a layer-by-layer deposition technique to create core-shell nanocomposites (Chen, T. Y. et al., J. Am. Ceram. Soc., 1998, 81: 140; Chen, T. Y. et al., Mater. Res. Innov., 1999, 2(6): 325-327).

A functionalized polyacrylamide (PAM) nanogel has also been produced for drug extraction (Liu, F. et al., “Polyacrylamide Microgels Synthesis, characterization and Modification for Overdosed Drug Detoxification,” in Particles 2002, Orlando, Fla., 2002). The modification strategy was based on control of the interactions among functional groups attached to the nanogels and the drug molecules. Functionalization involved hydrophobic moieties, ionic or both. Preliminary studies showed that the functionalized nanogel is able to extract amitriptyline (antidepressant) or bupivacaine (anesthetic) from aqueous and from saline solutions and to do so much better than the non-functionalized PAM nanogel. Comparisons were made on the basis of nanogel capacity, extraction efficiency and partition coefficient.

### EXAMPLE 6

### Characterization of Matrices

Monitoring of the opening up of polymers was conducted using surface plasmon resonance, the coiling/stretching of polymers using fluorescence spectroscopy and their dangling from a surface using electron spin resonance spectroscopy. Knowledge of dynamics is necessary to develop sensors that will respond to perturbations and therefore to design gel particles with rapid response capabilities geared towards commercial application.

A surface plasmon resonance spectroscope was used it to look at the dynamics of perturbation in real time. A technique of using aluminum oxide as a protective coating for the sensor surface was developed. Dynamics of polyacrylic acid perturbed with pH changes suggested unequal rates of extension and contraction—the former occurring on a slower time scale. The gel-like structure of the coiled species preventing rapid diffusion of the hydrdoxyl ions was proposed to be the cause (Chen, T. Y. et al., Mater. Res. Innov., 1999. 2((6)): 325-327). In a polymer-surfactant system, data using this technique suggested that the rapid binding stage was due to formation of double surfactant species and electrostatic repulsion rather than to collapse with formation of hydrophobic microdomains.

Physical Characterization. The synthesized nanogels are characterized in terms of their size (swelling phenomena), charge, solubility, solvent characteristics (ionic strength, pH, temperature and polarity etc.), and rheological properties. New testing procedures are designed to study the specific properties in the presence of toxins and ligands capable of binding with the novel nanogels.

Fluorescence probes are employed to sense the nature (micropolarity and microviscosity) (Chandar, P. et al., J. Colloid Interface Science, 1987, 117(1): 31-46; Kunjappu, J. T. et al., J. Phys. Chem., 1990, 94: 8464-8468; Campbell, A. & Somasundaran, P., J. Colloid Interface Science, 2000, 229: 257-260) of the environment of the nanogels. Probes which are sensitive to polarity or to local viscosity are employed. Both steady state and time resolved fluorescence are used as required by the system. For example, probes whose fluorescence quantum yields or spectral distributions are sensitive to the environment's polarity are used to sense and report the polarity of the nanogel particles. Fluorescence polarization provides information on the microviscosity of the environment. Information from such polarity viscosity probes is correlated with the results of experiments on delivery and uptake. The tendency of the toxin or the deliverable molecule to enter the gel particle is investigated. Change in fluorescence properties of the free aptamers and in nanogels upon ligand binding are quantified in terms of the intensity of the fluorescing radiation.

Electron spin resonance technique is employed to obtain the rotational correlation time of ESR probes (incorporated inside the gel particles during synthesis) (Chandar, P. et al., J. Phys. Chem., 1987, 91(1): 148-150). This gives information on the internal available space of the gel and the polarity (Malbrel, C. A. & Somasundaran, P. “Studies of Adsorbed Surfactant Layers at the Solid/Liquid Interface using Electron Spin Resonance,” in Third International Conference on Fundamentals of Adsorption, Sonthofen, West Germany, 1989) of such space, which is of use for the design of the uptake/delivery experiments. Electron spin resonance probes (typically nitroxides) are also employed to sense the microviscosity and micropolarity (Malbrel, C. A. et al., J. Colloid Interface Science, 1990, 137(2): 600-603) of the environment of the nanogels. The ESR technique allows the sensing of different environments and the verification of conclusions derived from the fluorescence experiments.

Atomic force microscopy (AFM) is used to study the topography of the gel particles by immobilizing them on a solid support. “Push-Pull” experiments using the AFM cantilever is conducted to obtain information on the modulus of elasticity of the nanogel.

Light scattering studies provides information on the size and the diffusion coefficient of the nanogels.

Analytical ultracentrifuge (AUC) studies is useful to determine the molecular weight distribution (Deo, N. et al., J. Dispersion Sci. and Tech., 2002. 23: 483-490) of the DNA aptamers, their complexes, and subsequently the aptamer loaded nanogels. The fluorescent tags in the aptamer primers assists in their detection through the built in UV-Visible spectroscopic detection system for the AUC.

Surface Plasmon Resonance (SPR) studies are employed to study the kinetics of binding (Sarkar, D. & Somasundaran, P., “Polymer Surfactant Kinetics Using Surface Plasmon Resonance Spectroscopy Dodecyltrimethylammonium Chloride/Polyacrylic Acid System,” Submitted to JCIS 2002) of ligands to the DNA aptamers. The ligands are immobilized on the gold sensor surface using well-known thiol chemistry. Subsequent exposure to the ligand of choice (glucose, insulin, Pb²⁺, etc.) enables studying the binding in real time. The binding rate constant and the affinity constant values thus obtained are used to choose the aptamer that is best suited for ligand binding in each case. For example, in the case where the ligand is a toxin and the goal is to remove the latter from the surrounding environment, the aptamer with the highest binding rate and the highest affinity constant is selected so as to remove the toxin as soon as possible and to prevent it from getting released again. But in the case where the ligand is a drug molecule that is to be delivered slowly over an extended period of time, the aptamer with a low rate constant and a medium affinity constant is selected for optimal delivery.

Adsorption isotherms, surface tension measurements and zeta potential measurements are conducted to obtain the thermodynamic parameters necessary for the modeling studies (Somasundaran, P., & Kumar, K., Colloid Surface A, 1997, 491-513).

Through optical activation, signal transduction is influenced in these gels. Cross linkers containing azo groups in the nanogel matrix are excited using the desired wavelength to initiate the photo-isomerization process. The ensuing change in the gel architecture is followed in real time.

Further experiments using AFM and SPR yields information on the nature of the response to perturbations, and the time scales of the response. Initial experiments are performed with simple polymer fragments and the results obtained are used to design nanogels for optimal performance.

Atomic force microscopy of nanogels immobilized on a solid support, and excited with an external perturbation vector provides a visual monitoring route to the physical changes taking place. In order to complement the lack of time resolution of atomic force microscope (AFM) experiments, surface plasmon resonance (SPR) is used (our SPR can monitor events at a time resolution of 30 milliseconds) to get real time quantitative information on the dynamic changes in the nanogel architecture.

The nanogels are first immobilized on the gold surface using thiol chemistry. They are subsequently excited with light at the excitation radiation of the fluorescent probes in the aptamers. Fluorescence induces conformational changes in the photo-isomerizable groups, allowing one to follow the induced conformational changes in the nanogel structure in real time.

In a separate experiment, a high intensity laser beam of a different wavelength is used to cause a localized temperature jump but without changing the conformation of the layer. This experiment shows how the polymer reacts to temperature changes, including how the polymer behaves at the junction of the hotspot and the surrounding cold region, i.e. in the presence of a temperature gradient.

These experiments are aimed at demonstrating the feasibility of coupling the specificity and flexibility of nucleic acids aptamers with nanogel technology for delivery of biologically relevant molecules. The results are also relevant to detection (sensors) and removal (sequestration) applications, and are applicable to a wide variety of ligands, limited only by the range of the chemistry of the aptamers, which is considerable.

### EXAMPLE 7

### Fluorescent Detection of Copper Using a Trypsin-Conjugated Copper-Activated Self-Cleaving DNA Aptazyme and a Solid-State Fluorogenic Pepteide Substrate

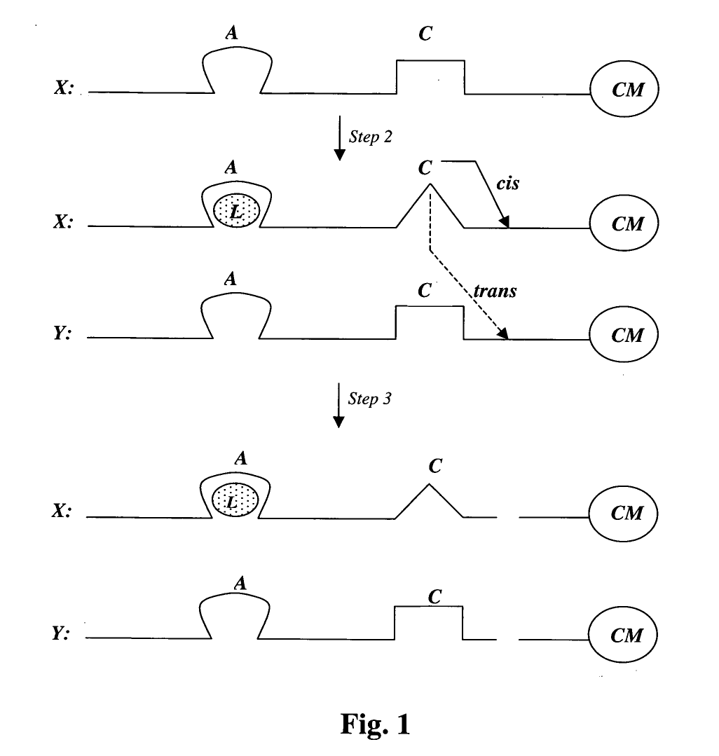

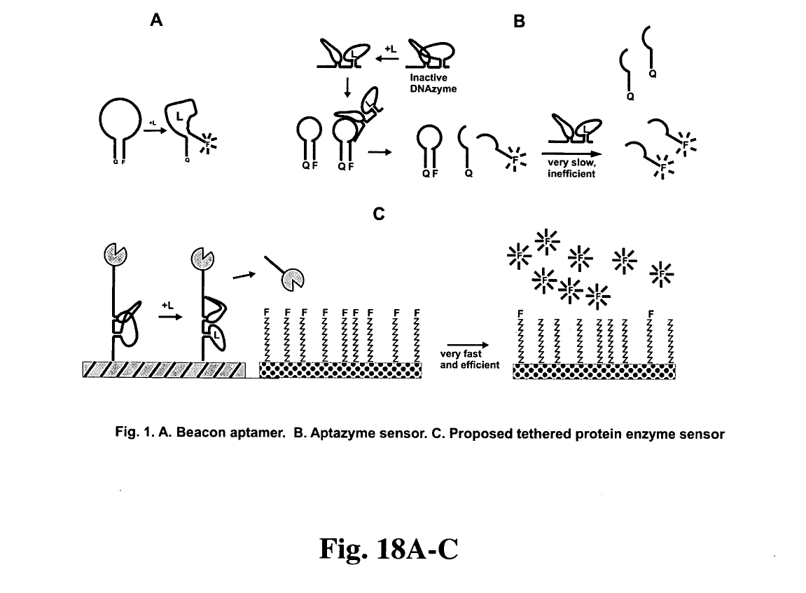

The invention provides compositions and methods to amplify the sensitivity of aptazymes serving as biosensors by coupling the activity of the aptazyme to that of traditional protein enzymes. This biosensor scheme is shown in FIG. 18C compared to two other aptamer-based biosensor schemes (FIGS. 1A and 1B). The protein enzymes are released from a solid-state, self-cleaving aptazyme in response to an environmental cue. FIG. 4 reiterates the overall scheme in more detail. This Example provides preliminary results that have been obtained utilizing the compositions and methods provided by the invention: 1) the proteolytic enzyme trypsin has been conjugated to a copper-activated self-cleaving DNA aptazyme and its release by exposure to copper has been effected; 2) a solid-state fluorogenic peptide substrate has been designed and synthesized that generates an optically detectable signal when exposed to trypsin; and 3) the two systems have been integrated such that exposure to copper results in a fluorescent signal. These 3 results are described below:

Construction an Avidin Bead-Biotinylated DNA-Trypsin Conjugate in which the Trypsin Moiety is Released by DNA Scission when Exposed to Copper

DNAzymes that cleave DNA substrates rather than ribozymes were used to avoid complications created by the chemical and enzymatic instability of RNA. Although several allosteric ribozymes have been described (reviewed in Breaker, R. R. Curr Opin Biotechnol 13, 31-9. (2002); Penchovsky, R. & Breaker, R. R. Nat Biotechnol 23, 1424-33 (2005); Swearingen, C. B. et al. Anal Chem 77, 442-8 (2005)), no allosteric DNAzyme had yet been engineered when the studies described in this Example were initiated. Recently, an allosteric DNAzyme that responds to adenosine has been constructed (Liu, J. & Lu, Y. Anal Chem 76, 1627-32 (2004)). However, that DNAzyme, which also requires Pb⁺⁺ ions for activity, needs at least one ribonucleotide at the cleavage site in its substrate sequence. As a result, the background cleavage of this substrate is high (Swearingen, C. B. et al. Anal Chem 77, 442-8 (2005)).

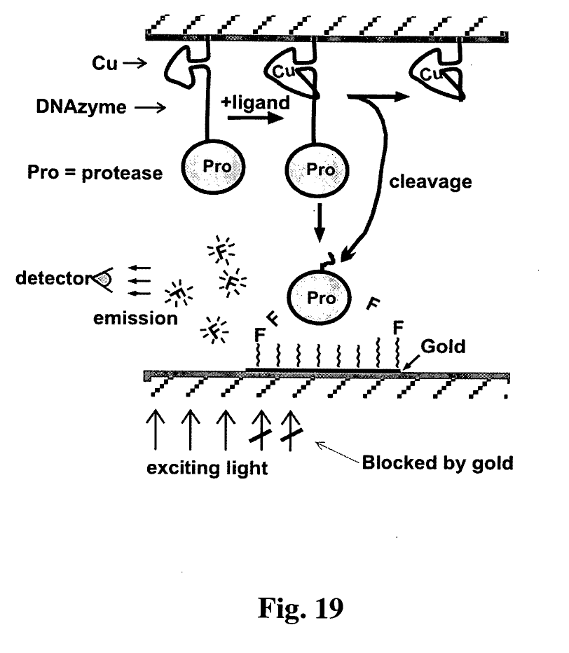

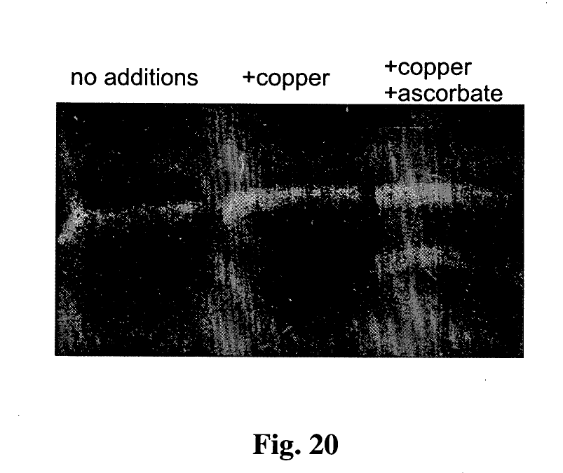

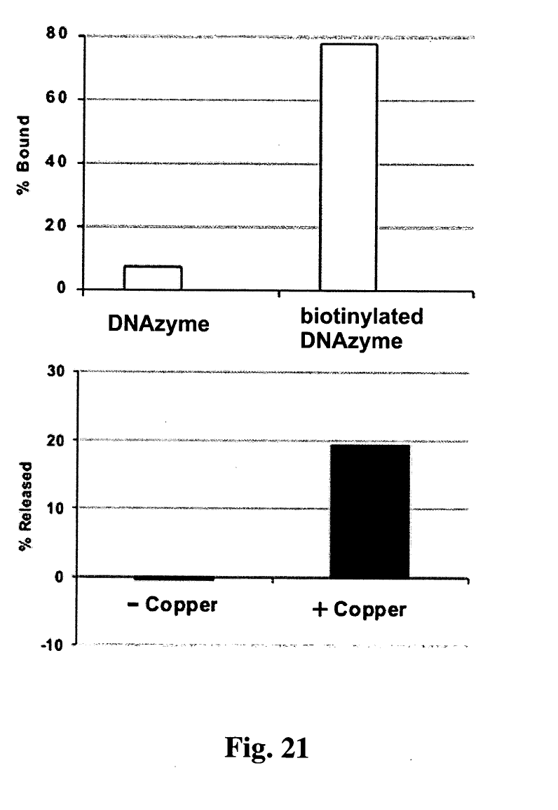

The enzyme release compositions and methods provided by this invention were studies using as a model system the copper-dependent DNAzyme with DNase activity described by Carmi and Breaker (Carmi, N., Balkhi, S. R. & Breaker, R. R. Proc Natl Acad Sci USA 95, 2233-7. (1998); Carmi, N. & Breaker, R. R. Bioorg Med Chem 9, 2589-600. (2001); Carmi, N., Shultz, L. A. & Breaker, R. R. Chem Biol 3, 1039-46. (1996)). This enzyme cleaves its substrate DNA sequence only in the presence of copper ions. The metal takes part in the catalytic reaction rather than functioning as an allosteric regulator, but this dependence is suitable for developing the enzyme release technology, as illustrated in FIG. 19. An attractive feature of this DNAzyme is its near absolute dependence on copper, with little cleavage (<0.01%) in the absence of the metal. The self-cleaving ability in a DNA molecule that contains both the DNAzyme and its substrate sequence was confirmed. The inclusion of the reducing agent ascorbate was necessary for efficient cleavage, suggesting that Cu⁺ rather than Cu⁺⁺ is the functional ion (FIG. 20). Ascorbate has been included in all mentions of copper below. Like Carmi et al., who assayed radioactively end-labeled DNA exclusively, it was found that these unlabeled molecules were not cut to completion, typically achieving 50% cleavage, suggesting an equilibrium between an active and an inactive conformational state. To construct a tripartite tethered reporter, the DNAzyme was synthesized with one end immobilized on a streptavidin bead and with a molecule of the protease trypsin bound to the other end. To determine whether the DNAzyme remained in an active conformation with these bulky appendages, the effect of immobilization on avidin beads was tested. The DNAzyme (as a 56-mer) was synthesized with a biotin moiety on its 3′ end (Invitrogen) and was then exposed to streptavidin beads (Sigma). Binding and release upon exposure to copper were measured by following absorbance at 260 nm. As shown in FIG. 21, most of the biotinylated DNA was bound to the beads. However, only 20% of the bound DNAzyme was self-cleaved after exposure to copper (0.1 mM), about ½ of what was expected based on the free DNAzyme. The studies described in this Example were conducted with this substantial if suboptimal yield. However, these results show that some of the DNAzyme molecule may be occluded on the surface of the bead or the streptavidin protein. A longer 3′ spacer may be added to improve the yield of cleaved molecules.

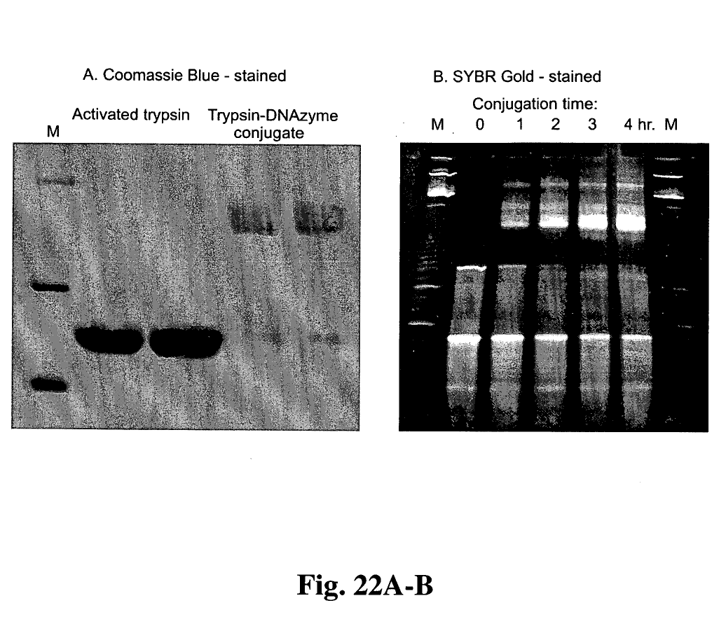

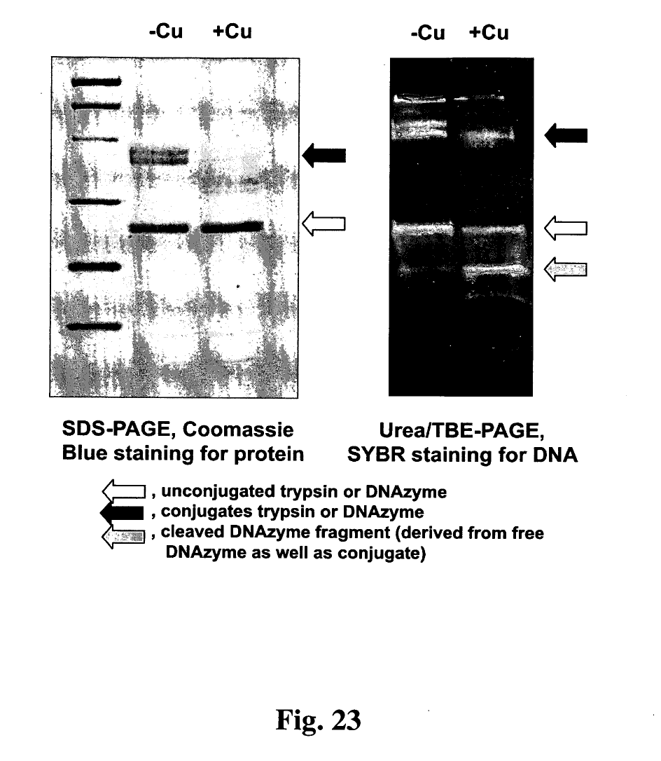

The DNAzyme was conjugated to trypsin by synthesizing the DNA with a 5′ aldehyde and reacting it with trypsin that had been activated at primary amino groups (lysines) by adding a hydrazine group. The aldehyde and hydrazine react under mild conditions to form a stable hydrazone linkage (Solulink reagents). The conjugate was produced at high yield, as evidenced by the gel electrophoresis shown in FIG. 22; the formation of the high molecular weight conjugate was detected either by following the trypsin protein by Coomassie Blue staining or the single-stranded DNAzyme by Sybr Gold staining (Invitrogen-Molecular Probes). When this conjugate was exposed to copper most of the DNA was cleaved, as evidenced by the substantial reduction of the high MW band (FIG. 23, black arrows).

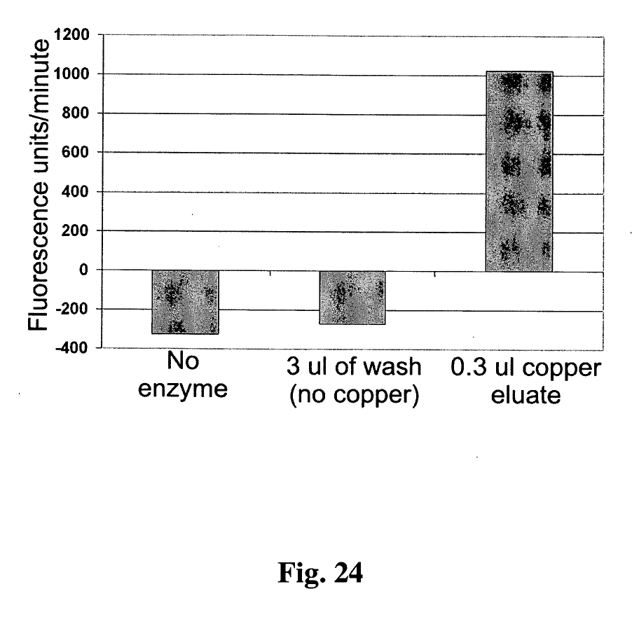

Studies were designed to test whether the fully tripartite complex can be cleaved to release trypsin tethered in this manner. A DNAzyme was synthesized with a 5′ aldehyde and a 3′ biotin group and bound to streptavidin beads. After thorough washing, the beads were exposed to 0.1 mM copper for 30 minutes and the trypsin in the supernatant was assayed spectrofluorimetrically using a custom-synthesized fluorogenic peptide (CAGSGSGPR-aminomethylcoumarin (SEQ ID NO:2)) substrate. The raw data from this experiment are shown in FIG. 24. Released trypsin was easily detected from 50 μl of beads exposed to copper, with no significant release in the absence of copper. Experiments can be designed to measure the yield of this release, the copper concentration dependence, and the time course. The release can be optimized by varying the pH and the exposure time. One can determine the optimizing effects of the addition of spacer sequences to the 5′ and 3′ ends of the original DNAzyme molecule.

Construction of an Immobilized Fluorescently Labeled Peptide Substrates Immobilized on Gold for the Ultrasensitive Measurement of Protease Action.

An immobilized substrate is a feature of this biosensor system that makes the generation of the fluorescent reporter dependent on the release of the tethered protease. Another advantage to making use of an immobilized fluorogenic substrate in an enzyme assay is that fluorogenic molecules are designed to harbor a fluorescent group in a quenched environment, such that the excitation energy is transferred to another part of the molecule rather than being released as light. Upon cleavage, this alternative transfer can no longer take place and upon excitation fluorescent light is emitted and measured. The quenched state of the fluorescent group is rarely zero, so what one observes is an increase from a background value to the value of the release molecule. This increment varies according to the degree of quenching but is typically about a factor of 20. The background value limits the sensitivity: if it is 5% then one would have to achieve cleavage of 5% of the substrate to effect a 2-fold signal increment. When an enzyme activity is generating the release, there are two opposing considerations: reducing the substrate concentration will increase the sensitivity by reducing the background, but as concentrations drop below the Km of the enzyme, reducing the substrate will compromise sensitivity due to a decrease in the overall cleavage rate. Reducing the substrate also reduces the total fluorescent signal capable of being generated, and thus the dynamic range.

The use of a fluorogenic substrate immobilized on an opaque surface, as provided by the invention, obviates this background problem, since the surface prevents excitation of the molecule in the first place. Once cleaved, the fluorescent moiety not only has its fluorescence unmasked but it has become free to diffuse away from the opaque surface into the area of the reaction vessel which the excitation beam is traversing. Thus the quenched background is never seen and most of the signal becomes detectable. The fluorogenic peptide CAGSGSGPR-AMC (AMC=7-amino-4-methyl-coumarin) was designed with cysteine at its amino terminus, and the sulfhydryl group in the side chain of this amino acid was used to immobilize the substrate on gold disks. The sulfhydryl-gold attachment technology is mature and widely used by those skilled in the art, the gold is opaque, and gold also serves as a potent quencher in its own right (Fan, C. et al. Proc Natl Acad Sci USA 100, 6297-301 (2003)).

One cm diameter gold disks were prepared and loaded with fluorogenic substrate. Adding trypsin to these disks results in the release of fluorescent AMC; the amount of released dye corresponded to a loading density of at least 18 pmoles/cm 2. Greater release may be achievable through the use of mercaptohexanol to minimize occlusion of the cleavage site on the gold surface (Herne, T. M. & Tarlov, M. J. J. Am. Chem. Soc. 119, 8916-8920 (1997)). Another experiment one can use to optimize the conditions is to prepare a rougher gold surface in an effort to increase loading density. However, the yield obtained in the present studies is high enough for these disks to be used as immobilized substrates. In their final configuration, one can determine dependence of activity on trypsin concentration, time, temperature, pH, and disk size to optimize performance. The limits of sensitivity of this immobilized substrate can be compared to its soluble form.

Detection of Copper Ions Using the Integrated System

The solid-state copper-sensitive DNA aptazyme was bound to streptavidin beads via a 3′ biotin group. Trypsin was conjugated to the aptazyme at its 5′ end via an aldehyde group. The trypsin substrate was also present in the well of a 12-well dish as a 9-amino acid synthetic peptide having cysteine at its amino terminus and arginine-amino-methyl-coumarin at its carboxyl end. The cleavage at arginine results in greatly increased fluorescence due to the freed coumarin. The peptide substrate was attached to a 1-cm gold-coated glass disk via the sulfhydryl group of the cysteine and placed at the bottom of the well of a 12-well dish containing 300 μl of reaction mixture. Copper was added (or not) at various concentrations and after 30 minutes the solution was withdrawn and centrifuged to remove the streptavidin beads. The fluorescence of the supernatant was then measured in a spectrofluorimeter.

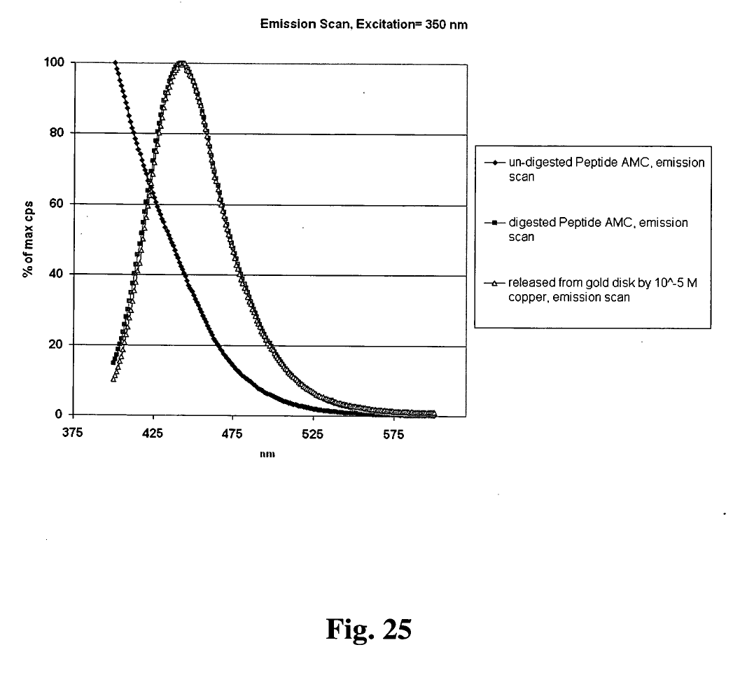

FIG. 25 shows the emission spectrum of the fluorescent material released by copper exposure. The spectrum corresponds to the cleavage product (coumarin) and not the substrate, showing that the observation is not simply the release of the entire substrate from the gold surface during the incubation. The same was true for the excitation spectrum.

The combined reaction involves two chemical processes proceeding simultaneously. Although the DNA cleavage and the action of trypsin have different pH and salt optima, conditions were found that allowed each to take place at close to their maximum rates.

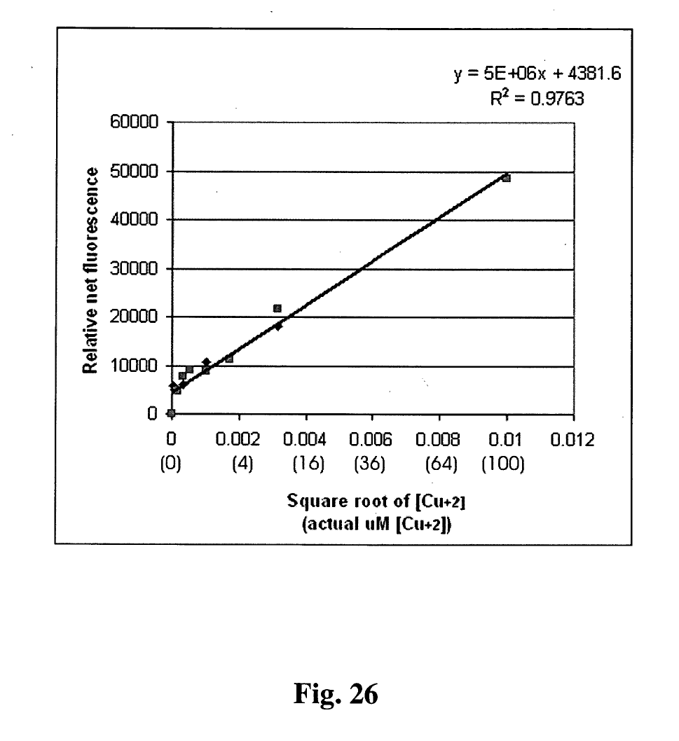

The ultimate experiment of this series is the use of this model system to detect copper. FIG. 26 shows the copper concentration dependence of the amplified combined reaction. The best fit to this relationship is a proportionality to the square root of the copper concentration, suggesting that either the copper or the released trypsin interacts with its solid state target with kinetics resembling that of a solute with a semipermeable membrane or ion channel. These results show that both parts of the amplification scheme are working in concert to allow detection of copper ions down to about 10⁻⁷ M, or 30 pmoles in a relatively large 300 μl reaction volume. The sensitivity is presently limited by the electronic background in the fluorescence measurements (a cooled photodetector could be used to further optimize the sensitivity), so one could improve the detection limits by miniaturization, using small reaction volumes and focused optics.

Methods

Copper-dependent DNAzyme cleavage assay and oligonucleotide PAGE. DNA oligonucleotides were synthesized at desalt purity by Invitrogen Corporation (Carlsbad, Calif.) and were used without further purification. The 46-mer copper-dependent self-cleaving DNAzyme sequence was flanked by five consecutive Ts at both 5′ and 3′ ends as flexible linkers:

5′-TTTTTGAATTCTAATACGACTCAGAATGAGTCTGGGCCTCTTTTTAAGAACTTTTT-3′. 5′-aldehyde modification of this 56-mer was used for trypsin conjugation and 3′-biotin modification for solid surface immobilization as further described below.

The DNA cleaving assay was performed essentially as described (Carmi, N., Balkhi, S. R. & Breaker, R. R. Proc Natl Acad Sci USA 95, 2233-7. (1998); Carmi, N. & Breaker, R. R. Bioorg Med Chem 9, 2589-600. (2001); Carmi, N., Shultz, L. A. & Breaker, R. R. Chem Biol 3, 1039-46. (1996)), with a few modifications. The DNAzyme was initially dissolved in 10 mM Tris HCl, pH 8.5 and further diluted into DNA cleavage buffer (50 mM HEPES, pH 7.0, 0.5 M NaCl, and 0.5 M KCl) at about 1 μM. Copper was supplied as CuCl₂ at indicated amounts in the DNA cleavage buffer together with 100 μM freshly prepared ascorbate (presence of ascorbate was necessary for maximum DNA cleavage activity). The mixture was incubated at room temperature for 15 minutes and a fraction of the reaction was subjected to denaturing polyacrylamide gel electrophoresis (PAGE). Electrophoresis was run using a 15% PAGE gel with 7M urea in TBE buffer (0.089 M Tris base, 0.089 M borate, and 25 mM EDTA). DNA (0.5 μg) oligonucleotide was mixed with 90% formamide in TBE buffer (v/v). The mixture was heated at 95° C. for five minutes and immediately chilled on ice. The gel was stained with SYBR Gold (Molecular Probes) and photographed according to the manufacturer's instructions.

The cleavage activity of trypsin-DNAzyme conjugate in solution was also monitored with SDS-PAGE. 12% SDS gels were stained with Coomassie blue and destained in 30% methanol and 10% acetic acid. The gel was soaked in 5% glycerol and air-dried.

For the immobilized DNAzyme cleavage assay, the 3′ biotin end-labeled DNAzyme was first added to streptavidin-agarose beads (Sigma, St. Louis, Mo.) in biotin binding buffer (1 mL of 50 mM Tris HCl, pH 7.4 and 1 M NaCl). The mixture was tumbled at room temperature for one hour. The beads were washed three times with 1 mL biotin binding buffer for 10 minutes each. The amount of bound oligonucleotide was calculated by the difference in absorption at 260 nm of pre- and post-incubation supernatants. The loaded beads were first incubated with DNA cleavage buffer with tumbling for 15 minutes at room temperature. The supernatant was collected as control. Subsequently, the beads were subjected to copper treatment (with 100 μM ascorbate) in 1 mL DNA cleavage buffer for 15 minutes at room temperature. The amount of cleavage product was calculated based on the 260 nm adsorption of the supernatant, correcting for the length of the DNAzyme and its cleavage product.

Trypsin Modification and Conjugation to the DNAzyme. Trypsin powder (2× crystallized and lyophilized, Worthington Biochemical Corporation, Lakewood, N.J.) was dissolved in PBS (100 mM sodium phosphate, pH 7.4 and 150 mM NaCl) and was further purified by gel filtration to single band purity on Coomassie blue-stained SDS-PAGE.

Trypsin and the DNAzyme were conjugated with HydraLink technology (Solulink Biosciences, San Diego, Calif.). Purified trypsin was modified with a 15-fold molar excess of succinimidyl-4-hydrazinonicotinate acetone hydrazone in PBS at room temperature for 4 hours. The reaction mixture was then buffer exchanged into conjugation buffer (100 mM MES, pH 4.7 and 150 mM NaCl) with Micro Bio-Spin columns (BioRad, Hercules, Calif.). Modified trypsin and 5′-aldehyde modified DNAzyme were mixed at a molar ratio of 2:1 and were incubated at room temperature for 3-5 hours. The conjugation mixture was used without further purification.

Fluorescence-based trypsin assay. Fluorogenic trypsin peptide substrate, CAGSGSGPR-AMC (Amino-Methyl-Coumarin) was synthesized by AnaSpec, Inc. (San Jose, Calif.). The peptide powder was dissolved in 0.5 mM in 10 mM Sodium Citrate, pH 6.2 for −80° C. storage. The peptide solution was incubated with 1 mM dithiothreitol at room temperature for at least 1 hour before use.

The trypsin assay was performed in a total of 300 μL of assay buffer (100 mM Tris HCl, pH 8.0 and 10 mM CaCl₂) with 5 μM peptide substrate at room temperature. Fluorescence was measured every five minutes for total of 30 minutes with a Jobin Yvon Fluorolog-3 spectrofluorimeter (excitation: 350 nm, emission: 440 nm, integration time: 1 second, slits: 1.0/0.5). These seven data points were plotted in Microsoft Excel and the linear slope from duplicate measurements was reported. Sequence-grade trypsin powder (Worthington Biochemical Corporation, Lakewood, N.J.) was dissolved in ice-chilled 1 mM HCl as a standard. Trypsin standards and samples were stored on ice before use. 1 M NaOH was used to cleanse the cuvette between trypsin samples, including standards, followed by 10 tap water rinses and 10 double-distilled water rinses.

Copper-dependent trypsin release. The DNAzyme doubly labeled with a 5′-aldehyde and a 3′-biotin group was mixed with modified trypsin at a 1:2 molar ratio in a total of 200 μL of conjugation buffer and the mixture was incubated at room temperature for 4 hours. Sodium chloride was then added to 1 M and a final volume of 1 mL.

Streptavidin-agrose beads were washed with 1 mL biotin binding buffer three times and were incubated with the conjugation product for one hour at room temperature. The loaded beads were washed with 1 mL biotin binding buffer three times (10 minutes per wash). One mL of DNAzyme cleavage buffer was added to the beads and incubated with tumbling for 15 minutes. The beads were centrifuged and the supernatant was taken as control.

The beads were then split equally into individual tubes for release; each release reaction contained 10 μL beads in bed volume (equivalent to 9 μmole of input DNAzyme). Various amounts of CuCl₂, together with 100 μM freshly-prepared ascorbate, were added to the beads in a final volume of 0.5 mL. After 15-minture incubation with tumbling, the beads were centrifuged and the supernatants transferred into fresh tubes stored on ice. Ten μL of supernatant from each reaction, together with trypsin standards, were sampled for trypsin activity. The trypsin activity in the release experiment was normalized to those of the trypsin standards.

While the foregoing invention has been described in some detail for purposes of clarity and understanding, these particular embodiments are to be considered as illustrative and not restrictive. It will be appreciated by one skilled in the art from a reading of this disclosure that various changes in form and detail can be made without departing from the true scope of the invention.

A fluorogenic molecule can comprise essentially any molecule that can fluoresce, for example, a fluorogenic dye. Specific examples of fluorogenic molecules include, for example, derivatives of fluorescein diphosphate; dimethylacridinone phosphate; p-hydroxyphenylacetic acid; 3-(p-hydroxyphenyl)propionic acid; 4-methylumbelliferyl phosphate; 6,8-difluoro-4-methylumbelliferyl phosphate; 4-methylumbelliferyl-β-D-galactopyranoside; fluorescein di-β-D-galactosidase; 4-methylumbelliferyl-galactoside 6-sulfate, GAAAPF-methylaminocoumarin, CAGSGSGPR-7-amino-4-methyl-coumarin or anthraniloyl-Lys-p-nitroanilide. Fluorogenic molecules can also include derivatized fluorogenic molecules. Derivatized fluorogenic molecules can be, for example, fluorogenic molecules that have attached immobilizing groups that tether the molecule to a solid surface. The derivatized fluorogenic molecule can also include, either as part of the immobilizing group or as a region between the immobilizing group and the fluorogenic part of the molecule, a region that can be specifically cleaved, either by a protein enzyme or by a nucleic acid cleaving region. The derivatized fluorogenic molecule can also be a fluorogenic peptide, where the peptide includes a region that can be specifically cleaved by a protein enzyme (without harming the fluorogenic potential of the peptide) and an immobilizing region that tethers the peptide to a solid surface. Examples of groups that can be used to immobilize a peptide include Cys residues to attach a peptide to a gold surface, and Lys residues that attach the peptide to an aldehyde activated surface.

The use of a fluorogenic substrate immobilized on an opaque surface, as provided by the invention, obviates this background problem, since the surface prevents excitation of the molecule in the first place. Once cleaved, the fluorescent moiety not only has its fluorescence unmasked but it has become free to diffuse away from the opaque surface into the area of the reaction vessel which the excitation beam is traversing. Thus the quenched background is never seen and most of the signal becomes detectable. The fluorogenic peptide CAGSGSGPR-AMC (AMC=7-amino-4-methyl-coumarin) was designed with cysteine at its amino terminus, and the sulfhydryl group in the side chain of this amino acid was used to immobilize the substrate on gold disks. The sulfhydryl-gold attachment technology is mature and widely used by those skilled in the art, the gold is opaque, and gold also serves as a potent quencher in its own right (Fan, C. et al. Proc Natl Acad Sci USA 100, 6297-301 (2003)).

Copper-dependent DNAzyme cleavage assay and oligonucleotide PAGE. DNA oligonucleotides were synthesized at desalt purity by Invitrogen Corporation (Carlsbad, Calif.) and were used without further purification. The 46-mer copper-dependent self-cleaving DNAzyme sequence was flanked by five consecutive Ts at both 5′ and 3′ ends as flexible linkers: 5′-TTTTTGAATTCTAATACGACTCAGAATGAGTCTGGGCCTCTTTTTAAGAACTTTTT-3′. 5′-aldehyde modification of this 56-mer was used for trypsin conjugation and 3′-biotin modification for solid surface immobilization as further described below.

Fluorescence-based trypsin assay. Fluorogenic trypsin peptide substrate, CAGSGSGPR-AMC (Amino-Methyl-Coumarin) was synthesized by AnaSpec, Inc. (San Jose, Calif.). The peptide powder was dissolved in 0.5 mM in 10 mM Sodium Citrate, pH 6.2 for −80° C. storage. The peptide solution was incubated with 1 mM dithiothreitol at room temperature for at least 1 hour before use.

What is claimed is:

1. A composition comprising:
(a) a nucleic acid comprising
(i) an aptamer region that specifically binds a ligand, and
(ii) a nucleic acid cleaving region, and
(b) a cargo molecule covalently linked to the nucleic acid,
wherein binding of the ligand to the aptamer results in release of the cargo molecule from the nucleic acid.

2. A composition comprising:
(a) a well;
(b) a first nucleic acid comprising an aptamer region that specifically binds a ligand, wherein the first nucleic acid is bound to the well;
(c) a second nucleic acid that is hybridized to the aptamer region;
(d) a cargo molecule covalently linked to the second nucleic acid;
wherein binding of the ligand to the aptamer results in separation of the first and second nucleic acids.

3. A composition comprising:
(a) a well;
(b) a nucleic acid comprising a stem-loop structure, wherein the stem comprises an aptamer region that specifically binds a ligand, and wherein the nucleic acid is bound to a surface in a first region in the well;
(c) a cargo molecule covalently linked to the nucleic acid;
(d) a plurality of reporter molecules bound to a surface in a second region in the well, wherein the surface in the second region comprises a metallic surface;
wherein binding of the ligand to the aptamer results in a dissolution of the stem-loop structure such that the nucleic acid is extended so the cargo molecule reaches into the second region in the well.

4. A composition comprising:
(a) a well;
(b) a nucleic acid comprising
(i) an aptamer region that specifically binds a ligand, and
(ii) a nucleic acid cleaving region,
wherein the nucleic acid is bound to a surface in a first region in the well;
(c) a cargo molecule covalently linked to the nucleic acid, wherein binding of the ligand to the aptamer results in release of the cargo molecule from the nucleic acid; and
(d) a plurality of fluorogenic molecules bound to a surface in a second region in the well, wherein the surface in the second region comprises a metallic surface.

5. A composition comprising:
(a) a well;
(b) a carbon nanotube comprising a transistor;
(c) a plurality of charged molecules bound to the exterior of the carbon nanotube;
(d) a nucleic acid comprising
(i) an aptamer region that specifically binds a ligand;
(ii) a nucleic acid cleaving region; and
(iii) a cargo molecule covalently linked to the nucleic acid;
wherein the nucleic acid is bound to the surface of the well;
wherein binding of the ligand to the aptamer results in diffusion of the cargo molecule to the charged molecules.

6. A composition comprising:
(a) a well;
(b) a carbon nanotube comprising a transistor;
(c) a plurality of substrate molecules bound to a first region on the exterior of the carbon nanotube;
(d) a nucleic acid comprising a stem-loop structure, wherein the stem comprises an aptamer region that specifically binds a ligand, and wherein the nucleic acid is bound to a surface to a second region on the carbon nanotube;
(e) a cargo molecule covalently linked to the nucleic acid;
wherein binding of the ligand to the aptamer results in a dissolution of the stem-loop structure such that the nucleic acid is extended so the cargo molecule reaches into the first region in the well.

7. A composition comprising:
(a) a nucleic acid comprising
(i) an aptamer region that specifically binds a ligand, and
(ii) a nucleic acid cleaving region, wherein the nucleic acid is linked to a matrix subunit thereby forming a matrix, and
(b) one or more cargo molecules contained within the matrix,
wherein binding of the ligand to the aptamer results in release of the one or more molecules from the matrix.

8. A composition comprising:
(a) a first nucleic acid comprising
(i) a first aptamer region that specifically binds a ligand, and
(ii) a first nucleic acid cleaving region, wherein binding of the ligand to the first aptamer region results in cleavage of a fragment from the first nucleic acid;
(b) a second nucleic acid comprising
(i) a second aptamer region that specifically binds the fragment, and
(ii) a second nucleic acid cleaving region, wherein the second nucleic acid is linked to a matrix subunit thereby forming a matrix; and
(c) one or more cargo molecules contained within the matrix,
wherein binding of the fragment to the second aptamer region results in release of the one or more molecules from the matrix.

9. A composition comprising:
(a) a nucleic acid comprising
(i) a first aptamer region; and
(ii) a second aptamer region that specifically binds a ligand; and
(b) a drug, wherein the drug is bound to the first aptamer region; and
wherein binding of the ligand to the second aptamer region results in release of the drug.

10. A composition comprising:
(a) a nucleic acid comprising
(i) an aptamer region that specifically binds a ligand,
(ii) a nucleic acid cleaving cleavage region, and
(iii) a terminal hairpin region, and
(b) a fluorophore covalently linked to the hairpin region,
wherein the hairpin structure quenches fluorescence of the fluorophore, wherein binding of the ligand to the aptamer results in cleavage of the hairpin structure, whereby the fluorescence of the fluorophore is no longer quenched.

11. The composition of claim 1, wherein the nucleic acid comprises DNA or RNA.

12. The composition of claim 1, wherein the nucleic acid cleaving region comprises a ribozyme or a DNAzyme.

13. The composition of claim 7, wherein the binding of the ligand to the aptamer region results in disassembly of the matrix.

14. The composition of claim 1, wherein the nucleic acid further comprises a recognition region recognized by the nucleic acid cleaving region.

15. The composition of claim 7, wherein the cargo molecule comprises a reporter enzyme or a therapeutic drug.

16. The composition of claim 15, wherein the reporter enzyme catalyzes a chromogenic, fluorogenic or luminogenic molecule.

17. The composition of claim 3, wherein the cargo molecule comprises an enzyme, wherein the enzyme is capable of releasing or cleaving the fluorogenic molecule.

18. The composition of claim 17, wherein the enzyme comprises a protease.

19. The composition of claim 5, wherein the cargo molecule comprises an enzyme, wherein the enzyme is capable of releasing or cleaving the charged molecule.

20. The composition of claim 19, wherein the enzyme comprises subtilisin, hyaluronidase, chitinase, cellulase, phospholipase C, or a DNA restriction enzyme.

21. The composition of claim 19, wherein the charged molecule comprises a peptide with a subtilisin cleavage site, hyaluronic acid, chitosan, carboxymethylcellulose, dipalmitoyl-phosphatidyl-inositol-diphosphate, or double stranded DNA.

22. The composition of claim 15, wherein the reporter enzyme comprises horseradish peroxidase, alkaline phosphatase, acid phosphatase, β-galactosidase, β-glucuronidase.

23. The composition of claim 16, wherein the chromogenic molecule comprises derivatives of 5-bromo-4-chloro-3-indolyl phosphate; 2,2′-azino-di[3-ethyl-benz-thiazoline sulfonic acid; 3,3′,5,5′-tetramethylbenzidine; o-phenylenediamine; p-nitrophenyl-phosphate; o-nitrophenyl-β-D-galactopyranoside; chloro-phenolic red-β-D-galactoopyranoside; or NADP glucose 6-phosphate.

24. The composition of claim 16, wherein the fluorogenic molecule comprises derivatives of fluorescein diphosphate; dimethylacridinone phosphate; p-hydroxyphenylacetic acid; 3-(p-hydroxyphenyl)propionic acid; 4-methylumbelliferyl phosphate; 6,8-difluoro-4-methylumbelliferyl phosphate; 4-methylumbelliferyl-β-D-galactopyranoside; fluorescein di-β-D-galactosidase; or 4-methylumbelliferyl-galactoside 6-sulfate.

25. The composition of claim 16, wherein the luminogenic molecule comprises derivatives of 1,2-dioxetanes; luminol; coeleterazines; luciferins; acridines; or metal ions.

26. The composition of claim 16, wherein the chromogenic, fluorogenic or luminogenic molecule is attached to a surface or a solid support.

27. The composition of claim 3, wherein the metallic surface comprises a gold surface.

28. The composition of claim 1, wherein the aptamer region comprises from about 15 to about 500 nucleotides, from about 15 to about 200 nucleotides, from about 15 to about 100 nucleotides or from about 40 to about 200 nucleotides.

29. The composition of claim 1, wherein the nucleic-acid cleaving region comprises from about 15 to about 500 nucleotides, from about 15 to about 200 nucleotides, from about 15 to about 100 nucleotides or from about 40 to about 200 nucleotides.

30. The composition of claim 7, wherein the matrix subunit comprises one or more of polyacrylamide, polysaccharide, polystyrene, polypropylene, polyethylene, polyurethane, polysiloxane, polymethyl methacrylate, polyvinyl alcohol, polyethylene, polyvinyl pyrrolidone, or any combination thereof.

31. The composition of claim 1, wherein the ligand comprises one or more of a chemical toxin, a pollutant, an allergen, a physiological indicator or any combination thereof.

32. The composition of claim 31, wherein the ligand is a bioterrorism agent.

33. The composition of claim 31, wherein the physiological indicator comprises glucose, calcium, uric acid, cholesterol, vitamin D, creatintine, bilirubin, triglycerides, hormones, or any combination thereof.

34. A composition comprising:
(a) a nucleic acid comprising
(i) a first aptamer region; and
(ii) a second aptamer region that specifically binds a ligand; and
(b) a drug, wherein the drug is bound to the first aptamer region; and
wherein binding of the ligand to the second aptamer region results in release of the drug.

35. The composition of claim 34, wherein the nucleic acid further comprises a third aptamer region, wherein the drug is bound to both the first and the third aptamer regions in the absence of ligand.

36. The composition of claim 34, wherein the drug comprises insulin.

37. The composition of claim 34, wherein the ligand comprises glucose.

38. A method for detecting the presence of a ligand comprising:
(i) contacting a sample the composition of claim 7 and
(ii) detecting whether or not cargo molecules are released from the matrix,
wherein detection of the molecules indicates presence of the ligand in the sample.

39. A method for detecting the presence of a ligand comprising:
(i) contacting a sample with the composition of claim 3, and
(ii) detecting whether there is an increase in fluorescence, wherein detection of the increase in fluorescence indicates presence of the ligand in the sample.

40. A method for detecting the presence of ligand comprising:
(i) contacting a sample with the composition of claim 16, and
(ii) detecting whether there is an increase in fluorescence, color, or chemilumenescence from the catalysis of a chromogenic, fluorogenic or luminogenic molecule, wherein detection of the increase in fluorescence, color, or chemilumenescence indicates presence of the ligand in the sample.

41. A method for delivering a molecule or a drug within a subject comprising:
(a) administering to the subject the composition of claim 1;
(b) contacting the composition with the ligand so as to release the molecule or the drug from the composition, thereby delivering the molecule or the drug within the subject.

42. The method of claim 41, wherein the ligand is glucose and the drug or molecule is insulin.

43. The method for detecting the presence of a ligand comprising
(i) contacting a sample with the composition of claim 5, and
(ii) detecting whether there is a change in electric current through the electric circuit, wherein detection of the change in electric current indicates presence of ligand in the sample.
